(19) United States
(12) Patent Application Publication
(10) Pub. No.: US 2011/0002944 A1
(43) Pub. Date: Jan. 6, 2011

(54) Compositions and Methods to Promote Neural Cell Growth

(75) Inventors: Guo-li Ming, Clarksville, MD (US); Eyleen Lay Keow Goh, Singapore (SG); Hongjun Song, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/223,883

(22) PCT Filed: Feb. 13, 2007
(86) PCT No.: PCT/US07/03950

§ 371 Date: Sep. 21, 2010

Related U.S. Application Data
(60) Provisional application No. 60/772,702, filed on Feb. 13, 2006.

(51) Int. Cl. A61K 39/395; A61K 38/00; A61K 31/7088; A61P 25/00; A61P 25/28; C12N 5/02; C12Q 1/02

(52) U.S. Cl. 424/172.1; 514/17.7; 514/44 R; 435/375; 435/29; 435/404

(57) ABSTRACT

The instant invention provides methods and compositions for promoting neural cell growth by inhibiting myelin associated glycoprotein (MAG)-induced inhibition of axonal regeneration. The invention relates to methods for identifying agents that inhibit MAG-induced inhibition of neural cell growth. Methods for inhibiting myelin associated glycoprotein (MAG)-induced inhibition of neural cell growth in a patient, methods for treating or preventing a MAG-induced disease or disorder of the CNS or PNS, comprising the step of administering at least one of the compositions according to this invention are provided. The invention includes kits comprising one or more agents identified according to the invention.

### CROSS REFERENCE TO RELATED APPLICATIONS & INCORPORATION BY REFERENCE

This application claims the benefit of U.S. Provisional Application No. 60/772,702 filed Feb. 13, 2006. The entire contents of the aforementioned application is hereby incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List before the paragraphs, or in the text itself; and, each of these documents or references (“herein-cited references”), as well as each document or reference cited in each of the herein-cited references (including any manufacturer's specifications, instructions, etc.), is hereby expressly incorporated herein by reference.

### GOVERNMENT SUPPORT

The following invention was supported at least in part by NIH Grant No.: NS048271. Accordingly, the government may have certain rights in the invention.

### BACKGROUND OF THE INVENTION

Nerve cell function depends upon appropriate contacts between the neuron and other cells in its immediate environment. These cells include specialized glial cells, oligodendrocytes in the central nervous system (CNS), and Schwann cells in the peripheral nervous system (PNS), which ensheathe the neuronal axon with myelin, is an insulating structure of multi-layered membranes.

Myelin is required for efficient nerve impulse conduction, but has other profound biological effects. The inability of nerves to regenerate after CNS injury in adults may be due largely to the axon's inability to grow when in contact with CNS myelin. Central nervous system injuries are particularly devastating because, unlike peripheral nerves, central nerves do not regenerate, so that central nervous system damage is usually permanent.

While CNS nerve cells have the capacity to regenerate after injury, molecules that are found in their local environment inhibit them from doing so. One such molecule is on myelin, and is referred to as myelin-associated glycoprotein (MAG). Thus, it would be of desirable therapeutic value to identify molecular factors which interfere with the ability of MAG to inhibit nerve growth. Knowledge of those factors would form a basis for developing drug therapies to interfere with MAG's inhibitory effects, and thus enhance the regeneration of nerves. Further, it would be useful to block the inhibitors of axonal regeneration for treating patients, for example patients with nervous system injuries where neural regeneration is a problem. One consideration in the design of such MAG-targeted therapies, however, is the dual role MAG plays as an inhibitory factor in neural regeneration in injury conditions or disease pathology, and its physiological role in maintaining the stability of intact axons. Thus, the specificity and control of the targeted therapy is an important consideration. Accordingly, there remains a need in the art to develop compositions and methods to inhibit MAG-induced failure of axonal regeneration and promote MAG-dependent axonal stability.

### SUMMARY OF THE INVENTION

As described below, the present invention provides methods and compositions for promoting neural cell growth by inhibiting myelin associated glycoprotein (MAG)-induced inhibition of axonal regeneration. The invention further relates to methods for identifying agents that inhibit MAG-induced inhibition of neural cell growth.

In one aspect, the invention features a method for inhibiting myelin associated glycoprotein (MAG)-induced growth inhibition in a neural cell, the method comprising contacting a neural cell with an effective amount of a beta1-integrin inhibitor or focal adhesion kinase (FAK) inhibitor, thereby inhibiting myelin associated glycoprotein (MAG)-induced growth inhibition in neural cells.

In a particular embodiment, MAG-induced growth inhibition in the neural cell comprises inhibition of axonal regeneration.

In one embodiment, the method increases neural growth, axonal outgrowth, or axonal regeneration.

In one embodiment, the neural cell is contacted in vivo or in vitro.

In another embodiment, the myelin associated glycoprotein (MAG)-induced growth inhibition is associated with central nervous system (CNS), peripheral nervous system (PNS) trauma or disease.

In another embodiment, the trauma or disease is selected from the group consisting of aneurysm, stroke, spinal cord or brain injury, Alzheimer's disease, Parkinson's disease, Creutzfeldt-Jacob disease, kuru, Huntington's disease, multiple system atrophy, amyotropic lateral sclerosis, spinal muscular atrophy, and progressive supranuclear palsy.

In another embodiment, the method comprises administering a beta1-integrin inhibitor and a focal adhesion kinase (FAK) inhibitor.

In a particular embodiment, the beta1-integrin inhibitor or focal adhesion kinase (FAK) inhibitor is a small compound, blocking antibody, polypeptide, polynucleotide, dominant negative polypeptide, or fragment thereof.

In one aspect, the invention features a method for promoting neural cell growth, the method comprising contacting a neural cell with an effective amount of a beta1-integrin inhibitor or focal adhesion kinase (FAK) inhibitor, thereby promoting neural cell growth.

In one embodiment, the neural cell is in contact with MAG.

In another aspect, the invention features a method for promoting neural cell growth in the presence of MAG, contacting the neural cell with a therapeutically effective amount of a beta1-integrin inhibitor or focal adhesion kinase (FAK) inhibitor, thereby promoting the growth of the neural cell in the presence of MAG.

In one embodiment of the above-mentioned aspects, the neural cell is a central nervous system (CNS) or peripheral nervous system (PNS) cell.

In another aspect, the invention features a method for inhibiting myelin associated glycoprotein (MAG) induced growth inhibition, the method comprising identifying a patient suffering from or susceptible to MAG-induced growth inhibition and administering a beta1-integrin inhibitor or FAK inhibitor compound to the identified patient, thereby inhibiting MAG-induced growth inhibition.

In one embodiment, the method increases axonal regeneration, neural outgrowth, or axonal extension.

In one aspect, the invention features a method for promoting the growth of a neural cell, the method comprising identifying a patient suffering from or susceptible to a MAG-induced inhibition of axonal regeneration, and administering a beta1-integrin inhibitor or FAK inhibitor compound to the identified patient, thereby promoting the growth of neural cells.

In one aspect, the invention features a method of treating or preventing a MAG-induced disease or disorder of the nervous system, the method comprising identifying a patient suffering from or susceptible to a MAG-induced disease or disorder of the nervous system and administering to the identified patient a beta1-integrin inhibitor or FAK inhibitor compound, thereby treating the disease or disorder.

In one embodiment, the method increases neural growth, axonal outgrowth, or axonal regeneration.

In a particular embodiment, the neural cell is contacted in vivo or in vitro.

In one embodiment of any of the above-mentioned aspects, the myelin associated glycoprotein (MAG)-induced growth inhibition is associated with central nervous system (CNS), peripheral nervous system (PNS) trauma or disease.

In a further embodiment of the method, the trauma or disease is selected from the group consisting of aneurysm, stroke, spinal cord or brain injury, Alzheimer's disease, Parkinson's disease, Creutzfeldt-Jacob disease, kuru, Huntington's disease, multiple system atrophy, amyotropic lateral sclerosis, spinal muscular atrophy, and progressive supranuclear palsy.

In another embodiment of the methods, the method comprises administering a beta1-integrin inhibitor and a focal adhesion kinase (FAK) inhibitor.

In a particular embodiment of any of the above-mentioned aspects, the beta1-integrin inhibitor or focal adhesion kinase (FAK) inhibitor is a small compound, blocking antibody, polypeptide, polynucleotide, dominant negative polypeptide, or fragment thereof.

In one embodiment, the MAG-induced disease or disorder is due to an alteration in MAG signaling.

In a particular embodiment of the above-described aspects, the method further comprises the step of detecting neural growth, axonal regeneration, or axonal outgrowth. In another embodiment, the neural cell growth is detected by MAG-induced turning responses of axonal growth cones in neural cells.

In one embodiment, the neural cell growth is detected by neurological examination. In another embodiment, the neurological examination detects an increase in sensation, motor function, or other neural function.

In one embodiment, the MAG-induced disease or disorder of the nervous system consists of trauma, injury or damage to neural cells. In a further embodiment, the MAG-induced disease or disorder of the nervous system is selected from the group consisting of: aneurysms, strokes, Alzheimer's disease, Parkinson's disease, Creutzfeldt-Jacob disease, kuru, Huntington's disease, multiple system atrophy, amyotropic lateral sclerosis, spinal muscular atrophy, and progressive supranuclear palsy.

In one embodiment of the above-described aspects, the beta1-integrin inhibitor or FAK inhibitor is administered to the patient orally, by injection or by an implantable device.

In one embodiment, the implantable device is an implantable solid or semi-solid device selected from the group consisting of: a biodegradable matrix, a fiber, a pump, a stent, and an adsorbable gelatin. In a further embodiment, the injection or the implantable device is delivered directly to a treatment site. In a particular embodiment, the site is a site of PNS or CNS trauma, injury, or disease. In one aspect, the invention features methods for identifying an agent that inhibits MAG-induced growth inhibition in neural cells, the methods comprising, contacting a neural cell with a candidate agent, detecting altered neural cell growth; and selecting the agent as a candidate if neural cell growth is promoted; thereby identifying an agent that inhibits myelin associated glycoprotein (MAG)-induced growth inhibition.

In one aspect, the invention features methods for identifying an agent that promotes the growth of neural cells, the methods comprising contacting a neural cell population with a candidate agent, detecting altered neural cell growth, and selecting the agent as a candidate if neural cell growth is promoted; thereby identifying an agent that promotes the growth of neural cells.

In another aspect, the invention features methods for identifying an agent that reduces FAK or beta1 integrin activity, the methods comprising contacting a neural cell expressing FAK or beta1 integrin with a candidate agent, and detecting a reduction in activity.

In a particular embodiment, the reduction in FAK activity is detected by measuring FAK phosphorylation or an increase in neural growth. In a further embodiment, the reduction in beta1 integrin activity is detected by measuring an increase in neural growth.

In one embodiment, the method further comprises the step of identifying the agent as a beta1-integrin inhibitor or a FAK inhibitor.

In a further embodiment, the agent is selected from the group consisting of a small compound, antibody, polypeptide, antisense oligonucleotide, siRNA, aptamer, ribozyme and fragments thereof.

In one embodiment, the FAK inhibitor is a FAK-pY397 inhibitor. In another embodiment, the FAK inhibitor is a FAK-pY861 inhibitor. In still another particular embodiment, the FAK inhibitor is a FAK inhibitory antibody that blocks FAK phosphorylation at Y397 or Y861.

In a particular embodiment of the above-described aspects, the neural cell is selected from the group consisting of: a retinal ganglion (RG), cerebellar neuron, cortical neuron, hippocampal neuron, motor neuron and superior-cervical ganglion. In another particular embodiment, the neural cells are mammalian.

In another aspect, the invention includes pharmaceutically acceptable compositions comprising an agent that inhibits MAG-induced growth inhibition in neural cells, and a pharmaceutically acceptable carrier.

In one embodiment, the agent is identified according to the method of any one of the aspects as described herein.

In another aspect, the invention includes pharmaceutically acceptable compositions for increasing neural growth comprising a FAK inhibitor and/or a beta1 integrin inhibitor.

In one embodiment, the pharmaceutically acceptable composition further comprises a second therapeutic agent.

In another aspect, the invention includes kits comprising one or more agents that inhibits MAG-induced growth inhibition of neural cells and promotes the growth of neural cells, and instructions for use.

In one embodiment, the kit further comprises a second therapeutic agent, and instructions for use. In another embodiment, the kit comprises an agent, wherein the agent is a FAK inhibitor or a beta1 integrin inhibitor.

### DESCRIPTION OF THE DRAWINGS

FIG. 1 (A-D) shows an association between MAG and β1-integrin in primary hippocampal neurons. Panel (A) shows sequence alignment of the RGD motif in the F-strand of MAG (Siglec-4) and SnD1 (Siglec-1) from different species. Panels (B to D) are western blots that show the association between MAG and β1-integrin. Primary hippocampal cultures were treated with wild type MAG-Fc (ROD), a soluble chimeric form of MAG, or mutant MAG-Fc (KGE) in the presence or absence of echistatin (100 nM) or the hemagglutin tag Ha2/5 (0.5 or 2.0 μg/ml). Cell lysates were immunoprecipitated (IP) with antibodies raised against β1-integrin and subjected to immunoblotting (IB) for human Fc, or vice versa.

FIG. 2 (A-D) shows that β1-integrin functions in MAG-induced axonal growth cone repulsion of hippocampal neurons. Panels (A to C) are a series of photomicrograph images that show growth cone turning in a gradient of MAG (150 μg/ml in the pipette). Sample images show the axons of P5 rat hippocampal neurons in the gradient on the left and the axonal growth cones at the onset (0′) and at the end (30′) of the turning assay at a higher magnification. Right traces show sample trajectories of axons during the turning assay from 15 randomly selected neurons. Scale bar: 5 μm. Panel (D) is a graph of a summary of the growth cone turning angles under different conditions. Pharmacological reagents were preincubated for 30 minutes at 37 C and present throughout the turning assay with the following concentrations: eschistatin (100 nM); Ha2/5 (1 μg/ml); Control 1 gM (1 μg/ml). Data represent mean +SEM. Numbers associated with bars indicate the number of growth cones analyzed under each condition. “*” indicates significant difference (p<0.01, ANOVA).

FIG. 3 is a pair of graphs showing MAG-induced growth cone turning responses of embryonic hippocampal neurons and postnatal cerebellum neurons. Shown is the summary of turning angles for axonal growth cones of rat embryonic day 17 (E17) hippocampal neurons (A) and P5 cerebellar neurons (CB)(B) in a gradient of MAG (150 μg/ml in the pipette), with or without the presence of Ha2/5 (1.0 μg/ml). Values represent mean +SEM. Numbers associated with the bar graph indicate the number of growth cones analyzed. “*” indicates significant difference (p<0.01, ANOVA).

FIG. 4 (A-C) shows that oligodendrocyte-myelin glycoprotein (Omgp)-induced growth cone repulsion does not require β1-integrin function. The pair of images in panel (A) show growth cone turning of rat hippocampal neurons in a gradient of OMgp. The sample images show an axon of a P5 hippocampal neuron in an OMgp gradient (5 μg/ml in the pipette) at the onset (0′) and at the end (30′) of the turning assay. Scale bar: 10 μm. Right traces show sample trajectories of axons during the turning assay from 15 randomly selected neurons. Scale bars: 5 μm. The pair of images in panel (B) show growth cone turning in a gradient of OMgp in the presence of a blocking antibody to β1-integrin. Panel (B) is similar as in (A), except for the presence of Ha2/5 (1.0 μg/ml). The graph in panel (C) is a summary of the growth cone turning angles under different conditions. Eschistatin (100 nM) or Ha2/5 (1 μg/ml) was preincubated for 30 minutes at 37 C and was present throughout the turning assay. Values represent mean +SEM. Numbers associated with the bar graph indicate the number of growth cones analyzed. “*” indicates significant difference from the heat-inactivated (HI) OMgp (p<0.01, ANOVA).

FIG. 5 (A and B) are two graphs that show that Nogo Receptor (NgR) is dispensable for MAG-induced growth cone repulsion of hippocampal neurons. In panel (A), the graph shows MAG-induced growth cone turning after phosphatidylinositol specific phospholipase C(PI-PLC) treatment. Primary hippocampal neurons were pre-treated with PI-PLC (1 unit/ml) for 30 min at 37C, and then growth cones were examined in a gradient of MAG. Shown is the summary of turning angles of axons with or without the PI-PLC treatment. In panel (B), the graph shows MAG-induced growth cone turning of hippocampal neurons from NgR knockout mice. Shown is the summary of turning angles for axonal growth cones of P5 mouse hippocampal neurons derived from wild-type or NgR knockout (NgR KO) mice in a gradient of MAG (150 μg/ml in the pipette), in the presence or absence of Ha2/5 (1.0 μg/ml). Values represent mean +SEM. Numbers associated with the bar graph indicate the number of growth cones analyzed. “*” indicates significant difference (p<0.01; ANOVA).

FIG. 6 (A-G) shows that MAG-induced tyrosine phosphorylation of FAK is required for growth cone repulsion to MAG. Panels (A to C) are immunoblots showing that MAG induces FAK phosphorylation. Shown in (A) is the time course of FAK phosphorylation after MAG stimulation (2 μg/ml) of rat hippocampal neurons. Cell lysates were immunoprecipitated with anti-FAK antibodies and immunoblotted with the pY-20 antibody for phosphorylated tyrosine residues. Shown in (B) are experiments in the presence or absence of Ha2/5 (1.0 μg/ml) or echistatin (100 nM). Shown in (C) are experiments with the treatment of wild type WT-MAG (RGD) or mutant MAG (KGE). Panel (D) is an immunoblot showing that MAG induces FAK phosphorylation on residues Y397 and Y861. Cell lysates of hippocampal neurons after MAG stimulation were immunoprecipitated (IP) with anti-FAK antibodies and immunoblotted (IB) with tyrosine phosphorylation site-specific antibodies to FAK. Panels (E to G) show that phosphorylation of FAK on residues Y397 and Y861 is required for MAG-induced growth cone repulsion. Hippocampal neurons were transfected with GFP, WT-FAK-GFP (E), or a FAK mutant with mutations at two tyrosine residues 397 and 861, FAK-Y397/861F-GFP (F). Growth cones of GFP+ neurons were examined in a gradient of MAG (150 μg/ml in the pipette). Sample images and traces were similarly shown as in FIG. 2 (A to C). Scale bar: 5 μm. The graph in panel (G) shows a summary of turning angles. Values represent mean +SEM. Numbers associated with the bar graph indicate the number of growth cones analyzed. “*” indicates significant difference from the control (GFP transfected neurons; p<0.01, ANOVA).

FIG. 7 is an immunoblot showing the lack of interaction between β1-integrin and members of the known Nogo Receptor (NgR) receptor complex. Lysates of HEK293 cells were transfected with expression constructs for tagged NgR, p75, TROY, or Lingo-1. Flag, myc, and hemaglutinin (HA) tags were used. Flag-NgR, myc-p75, myc-TROY, or HA-Lingo-1, with or without MAG treatment, were immunoprecipitated (IP) with anti-β1-integrin antibodies and immunblotted (IB) for the respective components of the NgR receptor complex. Also shown are immunoblots for total cell lysates showing the expression of endogenous β1-integrin and transfected proteins, and reblots for Fc showing a strong association of MAG and β1-integrin.

FIG. 8 is an immunoblot showing MAG-induced FAK phosphorylation after PI-PLC treatment. Hippocampal neurons were treated with saline or PI-PLC (2 units/ml) for 30 min at 37 C, then stimulated with MAG (1 μg/ml) for 15 minutes. Cell lysates were immunoprecipitated (IP) with anti-FAK antibodies and immunoblotted (IB) with pY-20 antibody for phosphorylated tyrosine (pY) residues. The membrane was reblotted for FAK to show similar loading.

### DETAILED DESCRIPTION OF THE INVENTION

The instant invention provides methods and compositions for promoting neural cell growth by inhibiting myelin associated glycoprotein (MAG) inhibition of axonal regeneration. The invention also relates to methods for identifying agents that promote neural cell growth in the presence of inhibition by MAG. The invention also relates to methods for promoting MAG-dependent axonal stability and prevention of degeneration. Methods for inhibiting myelin associated glycoprotein (MAG)-induced inhibition of axon regeneration in a patient, methods for treating or preventing a MAG-induced disease or disorder of the CNS, that involve administering at least one of the compositions according to this invention are provided. The invention includes kits containing one or more agents identified according to the invention.

### DEFINITIONS

The following definitions will be useful in understanding the instant invention.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “alteration” as used herein is meant to refer to a change. An increase refers to a positive alteration. A decrease refers to a negative alteration.

The term “axonal regeneration” is meant to refer, in general, to nerve regeneration. Methods to assess axonal regeneration include, but are not limited to, measurement of axonal extension, axonal diameter, or myelin thickness. The term “beta1 ([3])-integrin” (also known as CD29) is meant to refer to a beta subunit of the integrin family that functions in cell adhesion. Integrins are a large family of heterodimeric transmembrane glycoproteins that attach cells to extracellular matrix proteins of the basement membrane or to ligands on other cells. Integrins contain large (α) and small (β) subunits of sizes 120-170 kDa and 90-100 kDa, respectively beta1 (β1)-integrin can form heterodimer pairs with at least nine different alpha subunits. β1-integrin signals through a number of different pathways, for example kinase signaling pathways such as protein kinase C, MAP kinase, src kinase. Integrin signaling cascades, in some embodiments, promote the growth of neural cells. Integrin signaling cascades, in some embodiments, regulate (MAG)-induced growth inhibition in neural cells, in certain embodiments, exemplary beta 1-integrins include NM_{—}133376, NM_{—}033669, and NM_{—}010578.

The phrase “beta 1 (β1-integrin inhibitor” or “inhibitor of beta1 (β1)-integrin” is intended to refer to agents that decrease or otherwise promote the growth of neural cells (e.g., promote axonal outgrowth). A beta1-integrin inhibitor may reduce the activity of beta1-integrin receptor by at least about 2-fold, 5-fold, 10-fold, 20-fold, 40-fold, or even 100-fold as compared to a control condition. A beta1-integrin inhibitor may reduce the activity of beta1-integrin receptor by at least about 5%, 10%, 15%, 20%, 30%, 40%, 50%, 60%, 70%, 80%, 90% or even 100% as compared to a control condition. Inhibition of beta1 integrin activity can be measured by measurement of the downstream effectors of the beta1-integrin signaling pathway. For example, kinase activity of a receptor, or phosphorylation status of a receptor are indicative of activity levels.

The term “central nervous system” or “CNS” includes all cells and tissue of the brain and spinal cord of a vertebrate.

The term “control” is meant to refer to an untreated or reference subject or sample used in an experiment for comparison purpose.

A “disease or disorder of the central nervous system (CNS)” is meant to refer to diseases or disorders affecting the brain or spinal cord. Exemplary diseases of the CNS include, but are not limited to, aneurysms, strokes, Creutzfeldt-Jacob disease, kuru, multiple system atrophy, brain diseases, amblyopia, brain diseases, metabolic, brain edema, brain injuries, diffuse cerebral sclerosis of schilder, encephalitis, encephalomalacia, epilepsy, headache disorders, hydrocephalus, intracranial hypertension, subdural effusion, encephalomyelitis-meningitis fatigue syndrome, chronic angelman syndrome strabismus-pneumocephalus, spinal cord diseases, including amyotrophic lateral sclerosis, muscular atrophy, spinal cord compression, and spinal cord injuries. Also included are degenerative neurological diseases, such as Alzheimer's disease, Huntington's disease and Parkinson's disease.

The term “fragment” is meant to refer to a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference

The term “Focal Adhesion Kinase” (FAK) is mean to refer to a focal complex associated protein tyrosine kinase that is located in the cytoplasm. Exemplary FAK polypeptides include, but are not limited to, the amino acid sequence provided at NCBI Reference No. AAA35819. FAK is a major mediator of integrin signaling pathways. FAK is recruited at an early stage to focal adhesions and mediates many downstream responses of extracellular matrix proteins.

The phrase “FAK inhibitor” or “inhibitor of FAK” is intended to refer to compounds that decrease or otherwise interfere with the activity of FAK under normal or disease conditions. A FAK inhibitor reduces the activity of FAK receptor by at least about 2-fold, 5-fold, 10-fold, 20-fold, 40-fold, or even 100-fold as compared to a control condition. A FAK inhibitor reduces the activity of FAK receptor by at least about 5%, 10%, 15%, 20%, 30%, 40%, 50%, 60%, 70%, 80%, 90% or even 100% as compared to a control condition. FAK inhibition in certain embodiments is a reduction in phosphorylation of FAK.

The phrase “in combination with” is intended to refer to all forms of administration that provide an agent that inhibits MAG-induced axonal degradation, an agent that promotes MAG-induced neural cell growth and a second therapeutic agent, and can include concurrent or sequential administration, in any order.

The term “inhibit” is intended to mean decrease, suppress, attenuate, diminish, arrest, or stabilize the development or progression of a disease, disorder or condition.

For example, in certain embodiments, the inhibition is an inhibition by an agent of MAG-induced axonal degeneration. An inhibition can be a 5-fold, 10-fold, 20-fold, 40-fold, or even 100-fold inhibition as compared to a control condition. An inhibition can be a decrease of at least about 5%, 10%, 15%, 20%, 30%, 40%, 50%, 60%, 70%, 80%, 90% or even 100% as compared to a control condition.

The term “myelin associated glycoprotein (MAG)” is mean to refer to a component of myelinated internodes formed by oligodendrocytes in the central nervous system (CNS) and by Schwann cells in the peripheral nervous system (PNS).

The term “MAG induced disease or disorder” is meant to refer to a disease or disorder of the nervous system that is caused by myelin associated glycoprotein (MAG)-induced growth inhibition in neural cells.

The term “neural cell” is meant to refer to a cell of the nervous system. Exemplary neural cells include cells of the central nervous system and cells of the peripheral nervous system. These cells include isolated retinal ganglion (RG), cerebellum neurons, hippocampal (HN), motor neurons (MN) and superior-cervical ganglion (SCG).

The term “neural cell growth” is meant to refer to any increase in the length of axon extension during a defined period of time. In certain preferred embodiments, neural cell growth can be assayed by the total length of neural extension. Neural cell growth can also be assayed by MAG-induced turning responses of axonal growth cones in neural cells.

As used herein, the term “obtain”, as in “obtaining an agent” is meant to include synthesizing, purchasing or otherwise attaining.

The term “peripheral nervous system” is meant to refer to any sensory neuron or motor neuron. Sensory neurons run from stimulus receptors that inform the CNS of the stimuli and motor neurons run from the CNS to the muscles and glands. The peripheral nervous system is subdivided into the sensory-somatic nervous system and the autonomic nervous system

The terms “prevent,” “preventing,” “prophylactic treatment” and the like are meant to refer to reducing the probability of developing a disorder or condition in a subject, who does not have, but is at risk of or susceptible to developing a disorder or condition.

The term “promote” or “promoting” is meant to refer to a positive alteration. Alterations can include about 2-fold, 5-fold, 10-fold, 20-fold, 40-fold, or even 100-fold. In exemplary embodiments, promoting is of MAG-induced neural cell growth.

The term “signal transduction” is meant to refer to a process during which stimulatory or inhibitory signals are transmitted into and within a cell to elicit an intracellular response. A molecule can mediate its signaling effect via direct or indirect interact with downstream molecules of the same pathway or related pathway(s).

A “subject,” “individual” or “patient” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The phrase “therapeutically effective amount” is used herein to mean an amount sufficient to prevent, correct and/or normalize an abnormal physiological response. In one aspect, a “therapeutically effective amount” is an amount sufficient to reduce by at least about 30 percent, more preferably by at least 50 percent, most preferably by at least 90 percent, a MAG-induced disease or disorder of the CNS, MAG-induced neural cell growth, or to promote neuronal extension of growth.

The term “turning response” is meant to refer to the growth of an axon away from the original direction of extension

### Myelin Associated Glycoprotein (MAG)

Myelin-associated glycoprotein (MAG) is a component of myelinated internodes formed by oligodendrocytes in the central nervous system (CNS) and by Schwann cells in the peripheral nervous system (PNS). In MAG −/− mice, myelinated axons are smaller than normal and undergo degeneration and loss over time (Pan et al., 2005; Yin et al., 1998), suggesting that MAG plays a role in maintaining stability of the axon shaft (Scheduler and Bartsch, 2000). MAG has also been shown to promote neurite outgrowth during the embryonic development, but it inhibits axonal regeneration in the adult CNS (DeBellard et al., 1996; Filbin, 2003; He and Koprivica, 2004; Johnson et al., 1989; McKerracher et al., 1994; Mukhopadhyay et al., 1994; Schafer et al., 1996; Tumley and Bartlett, 1998). Following damage to the adult CNS, disruption of the myelin sheath leads to the release of a soluble fragment containing the MAG extracellular domain, which possesses potent inhibitory activity for neurite outgrowth (Tang et al., 1997b).

Recent studies suggested that three myelin proteins, myelin-associated glycoprotein (MAG), Nogo-A and oligodendrocyte myelin glycoprotein (OMgp), collectively account for the majority of the inhibitory activity in CNS myelin (4-6). The inhibitory activity of MAG, OMgp and the extracellular domain of Nogo-A may be mediated by a common receptor complex that consists of the ligand-binding Nogo-66 receptor (NgR) and its signaling co-receptors p75/TROY and Lingo-1 (7-13). However, little is known about how signaling events occurring at the axonal membrane are translated into specific cytoskeletal rearrangements underlying inhibition of axon regrowth.

Recently, a receptor complex consisting of NgR, p75/TROY and Lingo-1 has been proposed to mediate the inhibitory activities of three major myelin-associated inhibitors: MAG, Nogo66 (an extracellular domain of NogoA), and oligodendrocyte myelin glycoprotein (OMgp)(Domeniconi et al., 2002; Fournier et al., 2001; Liu et al., 2002; Mi et al., 2004; Park et al., 2005; Shao et al., 2005; Wang et al., 2002a; Wang et al., 2002b; Wong et al., 2002). Signaling of this receptor complex inhibits neurite outgrowth by activation of RhoA and Rock (Filbin, 2003; Lee et al., 2003; Yiu and He, 2006). While certain classes of neurons from p75 knockout mice exhibit reduced responses to myelin inhibitors, neurons lack of NgRs are still inhibited by these factors (Niederost et al., 2002; Zheng et al., 2005). Thus, it is likely that an additional signaling mechanism(s) is used for transducing the signaling of MAG and possibly other myelin-associated inhibitors. In addition, it is unknown whether MAG affects neuronal growth cones and axon shafts through different signaling mechanisms.

### Beta1 (β1)-Integrin

Integrins are a superfamily of cell adhesion receptors, which exist as heterodimeric transmembrane glycoproteins. They are part of a large family of cell adhesion receptors which are involved in cell-extracellular matrix and cell-cell interactions. Integrins play roles in cell adhesion to the extracellular matrix (ECM), which, in turn, mediates cell survival, proliferation and migration through intracellular signaling. The receptors consist of two subunits that are non-covalently bound. Those subunits are called alpha and beta. The alpha subunits all have some homology to each other, as do the beta subunits. The receptors always contain one alpha chain and one beta chain and are thus called heterodimeric. Both of the subunits contribute to the binding of ligand. Eighteen alpha subunits and eight beta subunits have been identified, which heterodimerize to form at least 24 distinct integrin receptors.

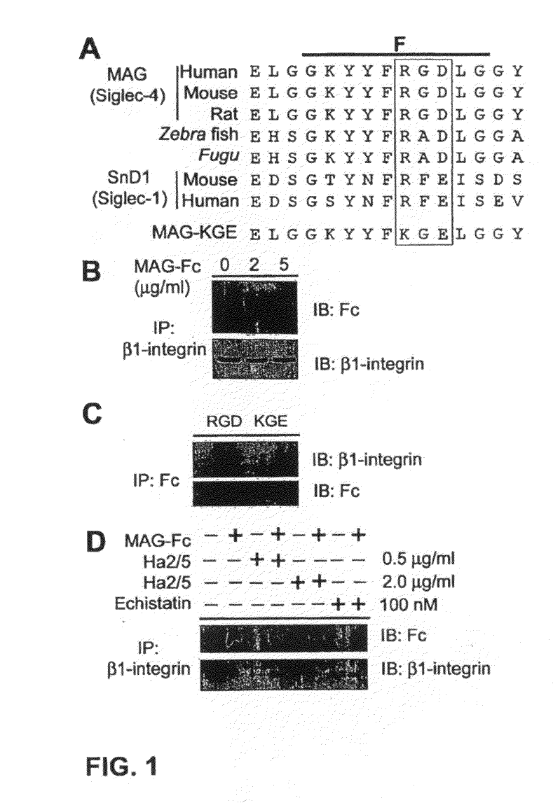

Integrins are heterodimeric receptors, consisting of β and β chains, for components of the extracellular matrix and for specific ligands (Hynes, 2002) and are important for cytoskeleton dynamics, cell adhesion and migration (Ridley et al., 2003). Emerging evidence has shown that integrins regulate axonal guidance and cell migration through different modes (Nakamoto et al., 2004). Integrins could act as, direct receptors for some guidance cues (Nikolopoulos and Gianotti, 2005). For example, netrin-1 binds integrins and regulates migration of epithelial cells (Yebra et al., 2003), while Sema7A promotes axon outgrowth of olfactory neurons through β1-integrins (Pasterkamp et al., 2003). Binding of guidance cues to their receptors has also been shown to alter the functions of integrins (Davy and Robbins, 2000; Serini et al., 2003). For example, Sema3A, signaling through neuropilin-1 and plexin A1, suppresses integrin functions that are important for adhesion and migration of endothelial cells (Serini et al., 2003). Many of the downstream signaling of guidance cues and integrins converges onto common pathways regulating cytoskeleton, thus integrins could also modulate the responsiveness of cells to guidance cues (Gomez et al., 2001; Stevens and Jacobs, 2002). Interestingly, human and rodent MAG (also called Siglec-4) contain the RGD tri-peptide, a characteristic binding motif recognized by integrin receptors containing β1 or β3 subunits (Ruoslahti, 1996; Ruoslahti and Pierschbacher, 1987)(FIG. 1A). Recent crystal structure analysis and modeling (May et al., 1998; Zaccai et al., 2003) suggest that the RGD motif in MAG (located within the F-strand, FIG. 1A) is not hidden from the protein surface as previously thought (Pedraza et al., 1990; Sadoul et al., 1990).

Focal Adhesion Kinase (FAK)

Focal adhesions are found at the cell membrane where the cytoskeleton interacts with proteins of the extracellular matrix. The clustering of integrins at these sites attracts a large complex of proteins and initiates intracellular regulatory processes, by which such events as cell migration and anchorage-dependent differentiation are controlled. Focal adhesion kinase (FAK) is a cytoplasmic protein tyrosine kinase that has been implicated to play an important role in integrin-mediated signal transduction pathways (Clark and Brugge, 1995; Schwartz et al., 1995; Parsons, 1996). FAK is recruited at an early stage to focal adhesions. FAK becomes activated and tyrosine phosphorylated in integrin-mediated cell adhesion and is colocalized with integrins and other cytoskeletal proteins in focal contacts in a variety of adherent cells (Schwartz et al., 1995).

Upon activation and autophosphorylation at Y397, FAK associates with another tyrosine kinase Src by binding to its SH2 (Src homology 2) domain (Chan, P.-Y., et all 994. A transmembrane-anchored chimeric focal adhesion kinase is constitutively activated and phosphorylated at tyrosine residues identical to pp 125FAK. J. Biol. Chem. 269: 20567-20574; Cobb, B. S. et al. 1994. Stable association of pp 60src and pp 59fyn with the focal adhesion-associated protein kinase, pp 125FAK. Mol. Cell. Biol. 14: 147-155; Schaller, M. D. et al. 1995. pp 125FAK-dependent tyrosine phosphorylation of paxillin creates a high-affinity binding site for Crk. Mol. Cell. Biol. 15: 2635-2645). Phosphorylation of Y925 of FAK by Src then recruits the Grb2 adaptor protein, which has been proposed to trigger downstream signaling pathways leading to activation of extracellular signal-regulated kinase (Erk) (Schlaepfer et al 1994. Integrin-mediated signal transduction linked to ras pathway by Grb2 binding to focal adhesion kinase. Nature. 372: 786-791; Schlaepfer et al. 1996. Evidence for in vivo phosphorylation of the Grb2 SH2-domain binding site on focal adhesion kinase by Src-family protein-tyrosine kinases. Mol. Cell. Biol. 16: 5623-5633). The FAK/Src complex has been shown to phosphorylate additional substrates including paxillin and p130cas, which have also been proposed to mediate downstream functions of FAK (Turner et al. 1994. Primary sequence of paxillin contains putative SH2 and SH3 domain binding motif and multiple LIM domains: identification of a vinculin and p125^{FAK} binding region. J. Cell Sci. 107: 1583-1591; Schaller et al. 1995. pp 125FAK-dependent tyrosine phosphorylation of paxillin creates a high-affinity binding site for Crk. Mol. Cell. Biol. 15: 2635-2645; Vuori et al. 1996.

Introduction of p130Cas signaling complex formation upon integrin-mediated cell adhesion: a role for Src family kinases. Mol. Cell. Biol. 16: 2606-2613). In addition, the phosphorylated Y397 has been mapped as the major binding site for the SH2 domains of the regulatory subunit (p85) of phosphatidylinositol 3-kinase (PI 3-kinase) and FAK association with PI 3-kinase in cell adhesion could trigger activation of PI 3-kinase and its signaling pathways (Chen et al. 1996. Phosphorylation of tyrosine 397 in focal adhesion kinase is required for binding phosphatidylinositol 3-kinase. J. Biol. Chem. 271: 26329-26334). Together, FAK interactions with these and potentially other proteins are believed to mediate FAK's functions in integrin-dependent signal transduction.

### Nervous System Disorders

The methods of the invention can be used to treat disorders of the central nervous system (CNS) or peripheral nervous system (PNS). The term “central nervous system” or “CNS” includes all cells and tissue of the brain and spinal cord of a vertebrate. Thus, the term includes, but is not limited to, neuronal cells, glial cells, astrocytes, cereobrospinal fluid (CSF), interstitial spaces, and the like. Regions of the CNS have been associated with various behaviors and/or functions. For example, the basal ganglia of the brain has been associated with motor functions, particularly voluntary movement. The basal ganglia is composed of six paired nuclei: the caudate nucleus, the putamen, the globus pallidus (or pallidum), the nucleus accumbens, the subthalamic nucleus and the substantia nigra. The caudate nucleus and putamen, although separated by the internal capsula, share cytoarchitechtonic, chemical and physiologic properties and are often referred to as the corpus striatum, or simply “the striatum.”

In certain embodiments, the MAG-induced disease or disorder of the CNS or PNS consists of trauma, injury or damage to neural cells, for instance in neurotoxic damage, or neurodegenerative diseases. The MAG-induced disease or disorder of the CNS can be selected from the group consisting of, but not limited to, aneurysms, strokes, Creutzfeldt-Jacob disease, kuru, Huntington's disease, multiple system atrophy, Brain Diseases/Amblyopia, Brain Diseases, Metabolic, Brain Edema, Brain Injuries, Diffuse Cerebral Sclerosis of Schilder, Encephalitis, Encephalomalacia, Epilepsy, Headache Disorders, Hydrocephalus, Intracranial Hypertension, Subdural

Effusion, Encephalomyelitis—Meningitis Fatigue Syndrome, Chronic Angelman Syndrome Strabismus—Pneumocephalus—Spinal Cord Diseases/Amyotrophic Lateral Sclerosis, Muscular Atrophy, Spinal, Spinal Cord Compression, and Spinal Cord Injuries and degenerative neurological diseases, including Alzeimer's disease and Parkinson's disease.

### METHODS OF THE INVENTION

Included in the invention are methods for inhibiting myelin associated glycoprotein (MAG)-induced growth inhibition in neural cells. The methods of the invention comprise contacting the neural cells with a therapeutically effective amount of a beta1-integrin inhibitor or focal adhesion kinase (FAK) inhibitor, and thereby inhibiting myelin associated glycoprotein (MAG) induced growth inhibition in neural cells. In certain embodiments, MAG-induced growth inhibition in neural cells comprises inhibition of axonal regeneration.

Also included in the invention are methods for promoting the growth of neural cells. The methods of the invention comprise contacting the neural cells with a therapeutically effective amount of a beta1-integrin inhibitor or focal adhesion kinase (FAK) inhibitor, thereby promoting myelin associated glycoprotein (MAG) induced growth inhibition in neural cells. In some embodiments, the neural cells are subjected to growth inhibition by MAG.

The invention also includes methods for promoting the growth of neural cells, wherein the neural cells are subjected to growth inhibition by MAG. The method comprises contacting the neural cells with a therapeutically effective amount of a beta1-integrin inhibitor or focal adhesion kinase (FAK) inhibitor, thereby promoting the growth of neural cells that are subjected to growth inhibition by MAG.

Neural cells according to the invention include, but are not limited to, isolated retinal ganglion (RG), cerebellum neurons, hippocampal (HN), motor neurons (MN) and superior-cervical ganglion (SCG).

Methods of Treating

As used herein, “treating” refers to preventing the onset of a MAG-induced disease or disorder of the CNS or PNS, and/or MAG-induced inhibition of neural cell growth, and/or reducing, delaying, or eliminating symptoms associated with a MAG-induced disease or disorder of the CNS or PNS or MAG-induced inhibition of neural cell growth. By “treating” is meant restoring the subject to the basal state as defined herein or to prevent a MAG-induced disease or disorder of the CNS or PNS, and/or MAG-induced inhibition of neural cell growth in a subject at risk thereof, or restoring the subject to the basal state. Alternatively, “treating” is meant to refer to arresting or otherwise ameliorating symptoms as defined herein.

As used herein delay means to retard or hinder the appearance of one of more symptoms of a MAG-induced disease or disorder of the CNS or PNS, or MAG-induced inhibition of neural cell growth as defined herein. A “delay” according to the invention can be at least 2 hours or more, for example, 2, 6, 12, 24, 36, 48, 60, 72 hours or 3 days or more, for example, 3, 4, 5, 10, 15, 20, 25, 30 or more days.

According to the methods of the invention, a MAG-induced disease or disorder of the CNS or PNS is treated, as defined herein, by administration of a beta1-integrin inhibitor or FAK inhibitor.

In certain embodiments, the invention includes methods for inhibiting myelin associated glycoprotein-induced growth inhibition. The methods comprise identifying a patient suffering from or susceptible to MAG-induced growth inhibition, and administering a beta1-integrin inhibitor or FAK inhibitor compound to the identified patient, thereby inhibiting MAG-induced growth inhibition. In certain embodiments, the MAG-induced growth inhibition in neural cells can comprise inhibition of axonal regeneration.

Also included in the invention are methods for promoting the growth of neural cells. The methods comprise identifying a patient suffering from or susceptible to MAG-induced inhibition of axonal regeneration, and administering a beta1-integrin inhibitor or FAK inhibitor compound to the identified patient, thereby promoting the growth of neural cells.

The invention includes methods of treating or preventing a MAG-induced disease or disorder of the nervous system. The methods comprise identifying a patient suffering from or susceptible to a MAG-induced disease or disorder of the nervous system, and administering to the identified patient a beta1-integrin inhibitor or FAK inhibitor compound, thereby treating the disease or disorder.

The MAG-induced disease of disorder of the nervous system can be any disease or disorder induced by myelin associated glycoprotein that occurs in the CNS or PNS. Exemplary disorders are described herein. In certain cases, the MAG-induced disease or disorder is due to an insufficient level of MAG signaling. For example, MAG signaling that is below a threshold level. In other cases, the MAG-induced disease or disorder is due to a lack of MAG signaling. In other certain cases, the MAG-induced disease or disorder is due to an elevated level of MAG signaling.

In certain preferred embodiments of the invention, neural cell growth can be assayed by the total length of neural extension. In other preferred embodiments, neural cell growth is assayed by MAG-induced turning responses of axonal growth cones in neural cells.

Neural cell growth can be assayed over a time period, for example 15 minutes, 30 minutes, 1 hour, 2 hours, 4 hours, 10 hours, 12 hours, 24 hours, 48 hours, 2 days, 4 days, 10 days, 15 days, 20 days, 30 days, 2 months, 4 months, 6 months, one year or more.

Another method of detecting neural cell growth is by neurological examination of the patient by the physician.

The present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a disease or disorder effecting the nervous system (e.g., a spinal cord injury, or neural degenerative disease) or symptom thereof. The method includes the step of administering to the mammal a therapeutic amount of an amount of an agent herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which an increase in neural growth (e.g., neural extension or axonal outgrowth) is required.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a disease or disorder effecting the nervous system (e.g., a spinal cord injury, or neural degenerative disease) in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### Screening Assays

The invention provides methods for promoting neural growth (e.g., neuronal regeneration, neurite extension, or axonal outgrowth) by altering MAG signalling via beta1 integrin or FAK. Accordingly, agents (i.e., small compounds, blocking antibodies, dominant negative mutations, polynucleotides, and the like) that disrupt a MAG signaling pathway are useful in the methods of the invention. Particularly useful are inhibitors of beta1 integrin and/or FAK inhibitors (e.g., any agent that reduces FAK phosphorylation or signaling). Methods of the invention are useful for the high-throughput low-cost screening of candidate agents that promote neural growth by altering (e.g., increasing or decreasing) MAG signaling via beta1 integrin or FAK. A candidate agent that specifically binds to MAG, beta1 integrin, or FAK is then isolated and tested for activity in an in vitro assay or in vivo assay for its ability to enhance neural growth (e.g., axonal outgrowth). One skilled in the art appreciates that the effects of a candidate agent on a neural cell is typically compared to a corresponding control cell not contacted with the candidate agent. Thus, the screening methods include comparing the outgrowth of a neural cell contacted by a candidate agent to the outgrowth of an untreated control cell.

In other embodiments, the expression or activity of MAG, beta1 integrin, and/or FAK in a cell treated with a candidate agent is compared to untreated control samples to identify a candidate compound that alters the expression or activity of MAG, beta1 integrin, or FAK in the contacted cell. Polypeptide expression or activity can be compared by procedures well known in the art, such as Western blotting, flow cytometry, immunocytochemistry, binding to magnetic and/or MAG, beta1integrin, or FAK-specific antibody-coated beads, in situ hybridization, fluorescence in situ hybridization (FISH), ELISA, microarray analysis, RT-PCR, Northern blotting, or colorimetric assays, such as the Bradford Assay and Lowry Assay.

In one example, one or more candidate agents are added at varying concentrations to the culture medium containing a neural cell. An agent that inhibits FAK or beta1 integrin activity is considered useful in the invention; such an agent may be used, for example, as a therapeutic to prevent, delay, ameliorate, stabilize, or treat an injury, disease or disorder where an increase in axonal outgrowth or a reduction in neural degeneration is desirable. Once identified, agents of the invention (e.g., beta1integrin, or FAK inhibitors) may be used to increase axonal outgrowth in a patient in need thereof).

In one embodiment, the effect of a candidate agent may, in the alternative, be measured at the level of FAK or beta1 integrin polypeptide production using the same general approach and standard immunological techniques, such as Western blotting or immunoprecipitation with an antibody specific for FAK or beta1 integrin. For example, immunoassays may be used to detect or monitor the expression of FAK or beta1integrin.

In one embodiment, the invention identifies a polyclonal or monoclonal antibody (produced as described herein) that is capable of binding to and reducing the activity of FAK or beta1 integrin. A compound that reduces the expression or activity of a FAK or beta1 integrin polypeptide is considered particularly useful. Again, such a molecule may be used, for example, as a therapeutic to enhance neural growth (e.g., axonal outgrowth or regeneration).

Alternatively, or in addition, candidate agents may be identified by first assaying those that specifically bind to and reduce the activity of a FAK or beta1 integrin polypeptide and subsequently testing their effect on axonal outgrowth as described in the Examples (e.g., by assaying turning or an increase in neurite length). In one embodiment, the efficacy of a candidate agent is dependent upon its ability to interact with the FAK or beta1 integrin polypeptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate neural outgrowth may be assayed by any standard assays (e.g., those described herein) Potential FAK or beta1 integrin inhibitors include organic molecules, peptides, peptide mimetics, polypeptides, nucleic acid ligands, aptamers, and antibodies that bind to a FAK or beta1 integrin polypeptide and inhibit its activity. Methods of assaying FAK or beta1 integrin activity include measuring FAK kinase activity or axonal outgrowth.

In one particular example, a candidate compound that binds to a FAK or beta1 integrin polypeptide may be identified using a chromatography-based technique. For example, a recombinant FAK or beta1 integrin polypeptide of the invention may be purified by standard techniques from cells engineered to express the polypeptide, or may be chemically synthesized, once purified the peptide is immobilized on a column. A solution of candidate agents is then passed through the column, and an agent that specifically binds the FAK or beta1 integrin polypeptide or a fragment thereof is identified on the basis of its ability to bind to FAK or beta1 integrin polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). In addition, these candidate agents may be tested for their ability to modulate axonal outgrowth or regeneration (e.g., as described herein). Agents isolated by this approach may also be used, for example, as therapeutics to treat or prevent the onset of a disease or disorder characterized by a reduction in axonal outgrowth or where an increase in outgrowth or regeneration is desirable (e.g., spinal cord injury or neural degenerative diseases). Agents that are identified as binding to a FAK or beta1 integrin polypeptide with an affinity constant less than or equal to 1 nM, 5 nM, 10 nM, 100 nM, 1 mM or 10 mM are considered particularly useful in the invention. Such agents may be used, for example, as therapeutics to enhance neural growth.

Test Compounds and Extracts

In general, agents that inhibit FAK or beta1 integrin polypeptides are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known those known as therapeutics for the treatment of spinal cord injury or neural degeneration. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N. H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have FAK or beta1 integrin polypeptide binding and/or inhibiting activity further fractionation of the positive lead extract is necessary to isolate molecular constituents responsible for the observed effect. Thus, the goal of the extraction, fractionation, and purification process is the careful characterization and identification of a chemical entity within the crude extract that enhances neural growth, axonal outgrowth or regeneration. Methods of fractionation and purification of such heterogenous extracts are known in the art. If desired, compounds shown to be useful as therapeutics are chemically modified according to methods known in the art.

### Pharmaceutical Compositions

The invention provides for compositions comprising an agent that inhibits MAG-induced axonal degradation, and a pharmaceutically acceptable carrier. The invention also provide's for compositions comprising an agent that inhibits MAG-induced inhibition of neural cell growth, and a pharmaceutically acceptable carrier. Further, the invention provides for compositions comprising any combination of agent that inhibits MAG-induced inhibition of neural cell growth and a second therapeutic agent.

Beta 1-integrin and FAK inhibitors of the invention can include blocking antibodies, siRNA/RNAi, shRNA, microRNA, antisense oligonucleotides, ribozymes, aptamers, chemical agents, phosphorylation inhibitors, or small molecules.

In certain embodiments, by introducing deletions or other mutations into the nucleic acids encoding beta1-integrin of FAK, or by using suitable fragments, it is possible to generate sequences encoding dominant-negative peptides or polypeptides. Treatment of diseases as described herein using said nucleic acid molecules can be achieved in different ways familiar to the person skilled in the art. For example, the isolated nucleic acid molecules may be inserted downstream of a strong promotor to overexpress the corresponding protein or polypeptide, for example beta1-integrin or FAK. Overexpression of the protein or polypeptide may lead to suppression of the endogenous protein's biological function. By introducing deletions or other mutations into the nucleic acids, or by using suitable fragments, it is possible to generate sequences encoding dominant-negative peptides or polypeptides. Such dominant-negative peptides or polypeptides can inhibit the function of the corresponding endogenous protein. Such dominant-negative peptides or polypeptides are useful in methods of treatment as described herein.

RNA molecules may be identified and used in a therapeutic application of the RNAi technique, particularly in humans or in human cells. An RNAi technique particularly suited for mammalian cells makes use of double-stranded RNA oligonucleotides known as “small interfering RNA” (siRNA). SiRNA molecules can be used for the therapeutic silencing of the expression of the genes of the invention comprising nucleic acid sequences of the invention, in mammalian cells, particularly in human cells, particularly for the therapy of diseases as described herein.

The inhibition of a specific target gene in mammals is achieved by the introduction of an siRNA-molecule having a sequence that is specific (see above) for the target gene into the mammalian cell. SiRNAs may be identified as inhibitors of beta1-integrin or FAK. siRNAs comprise a first and a second RNA strand, both hybridized to each other, wherein the sequence of the first RNA strand is a fragment of one of the sequences of the invention and wherein the sequence of the second RNA strand is the antisense-strand of the first RNA strand. The siRNA-molecules may possess a characteristic 2- or 3-nucleotide 3′-overhanging sequence. Each strand of the siRNA molecule preferably has a length of 19 to 31 nucleotides. siRNAs can be introduced into the mammalian cell by any suitable known method of cell transfection, particularly lipofection, electroporation or microinjection. SiRNA oligonucleotides can be generated and hybridized to each other in vitro or in vivo according to any of the well-known RNA synthesis methods. For instance, the nucleic acid molecule is contained in at least one nucleic acid expression vector which is capable of producing a double-stranded RNA-molecule comprising a sense-RNA-stand and an antisense-RNA-strand under suitable conditions, wherein each RNA-strand, independently from the other, has a length of 19 to 31 nucleotides.

In this method (also described in Tuschl, Nature Biotechnology, Vol. 20, pp. 446-448), vector systems capable of producing siRNAs instead of the siRNAs themselves are introduced into the mammalian cell for down-regulating gene expression. The preferred lengths of the RNA-strands produced by such vectors correspond to those preferred for siRNAs in general, and known in the art.

An alternative to transfecting cells with chemically synthesized siRNAs are DNA-vector-mediated mechanisms to express substrates that can be converted into siRNA in vivo. In the first approach the sense and antisense strands of the siRNA are expressed from different, usually tandem promoters. Alternatively, short hairpin (sh)RNAs are expressed and processed by Dicer into siRNAs. In general, chemically synthesized short interfering (si)RNA sequences that are effective at silencing gene expression are also effective when generated from short hairpin (sh)RNAs. However, the length of the stem and the size and composition of the loop are important for the efficiency of silencing.

The coding sequence of interest may, if necessary, be operably linked to a suitable terminator or to a poly-adenylation sequence. In the case of RNA, particularly siRNA, “coding sequence” refers to the sequence encoding or corresponding to the relevant RNA strand or RNA strands.

Further, the vector may comprise a DNA sequence enabling the vector to replicate in the mammalian host cell. Examples of such a sequence—particularly when the host cell is a mammalian cell—is the SV40 origin of replication.

A number of vectors suitable for expression in mammalian cells are known in the art and several of them are commercially available. Some commercially available mammalian expression vectors which may be suitable include, but are not limited to, pMC1neo (Stratagene), pXT1 (Stratagene), pSG5 (Stratagene), pcDNAI (Invitrogen), EBO-pSV2-neo (ATCC 37593), pBPV-1(8-2) (ATCC 37110), pSV2-dhfr (ATCC 37146). Preferred are all suitable gene therapeutic vectors known in the art.

In a particularly preferred embodiment of the invention the vector is a retroviral vector. Retroviruses are RNA-viruses possessing a genome that after the infection of a cell, such as a human cell, is reversely transcribed into DNA and subsequently is integrated into the genome of the host cell. Retroviruses enter their host cell by receptor-mediated endocytosis. After the endocytosis into the cell the expression of the retroviral vector may be silenced to ensure that only a single cell is infected. The integration of the viral DNA into the genome is mediated by a virus-encoded protein called integrase, wherein the integration locus is not defined. Retroviral vectors are particularly appropriate for their use in gene therapeutic methods, since their transfer by receptor-mediated endocytosis into the host cell, also known to those skilled in the art as “retroviral transduction” is particularly efficient. A person skilled in the art also knows how to introduce such retroviral vectors into the host cell using so called “packaging cells”.

In another particularly preferred embodiment of the invention, the vector is an adenoviral vector or a derivative thereof. Adenoviral vectors comprise both replication-capable and replication-deficient vectors. The latter include vectors deficient in the E1 gene.

The recombinant vector is preferably introduced into the mammalian host cells by a suitable pharmaceutical carrier that allows transformation or transfection of the mammalian, in particular human, cells. Preferred transformation/transfection techniques include, but are not limited to, liposome-mediated transfection, virus-mediated transfection and calcium phosphate precipitation.

In a preferred embodiment, the invention relates to the use of a vector system capable of producing siRNAs as defined above, wherein the nucleic acid corresponding to the siRNA is contained in at least one nucleic acid expression vector comprising a first expression cassette containing the nucleic acid corresponding to the sense-RNA-strand under the control of a first promoter and a second expression cassette containing the nucleic acid corresponding to the antisense-RNA-strand under the control of a second promoter.

In the above mentioned vector system, the vector comprises two individual promoters, wherein the first promoter controls the transcription of the sense-strand and the second promoter controls the transcription of the antisense strand (also described in Tuschl, Nature Biotechnology, Vol. 20, pp. 446-448). Finally the siRNA duplex is constituted by the hybridisation of the first and the second RNA-strand.

MicroRNAs (miRNAs) are evolutionarily conserved small non-protein-coding RNA gene products that regulate gene expression at the post-transcriptional level. In animals, mature miRNAs are ˜22 nucleotides long and are generated from a primary transcript through sequential processing by nucleases belonging to the RNAseIII family.

An alternative to transfecting cells with chemically synthesized siRNAs are DNA-vector-mediated mechanisms to express substrates that can be converted into siRNA in vivo. In the first approach the sense and antisense strands of the siRNA are expressed from different, usually tandem promoters. Alternatively, short hairpin (sh)RNAs are expressed and processed by Dicer into siRNAs. In general, chemically synthesized short interfering (si)RNA sequences that are effective at silencing gene expression are also effective when generated from short hairpin (sh)RNAs. However, the length of the stem and the size and composition of the loop are, important for the efficiency of silencing.

Other inhibitors include small molecules. Small molecules are molecules which are not proteins, peptides antibodies or nucleic acids, and which exhibit a molecular weight of less than 5000 Da, preferably less than 2000 Da, more preferably less than 2000 Da, most preferably less than 500 Da. Such small molecules may be identified in high throughput procedures/screening assays starting from libraries. Such methods are known in the art. Suitable small molecules can also be designed or further modified by methods known as combinatorial chemistry.

In some cases suitable antibodies can be used to inhibit the biological function of target proteins they bind to. Blocking antibodies of beta1-integrin and FAK thus can be identified according to the invention. In certain embodiments, an antibody that blocks, for example, the phopsphorylation of FAK may be used to block FAK activity. Antibodies that block phosphorylation at residues Y392 and/or Y925 are particularly desirable. Likewise, a beta-1 integrin antibody may be used that prevents receptor dimerization and/or downstream signaling events. Inhibition may occur by inducing conformational changes upon binding to the target protein. Another possibility is that the antibody binds two or more target proteins and prevents physical proximity to inhibit interaction of the target proteins.

Antibodies can be coupled covalently to a detectable label, such as a radiolabel, enzyme label, luminescent label, fluorescent label or the like, using linker technology established for this purpose. Labeling is particularly useful for diagnostic purposes (see below) or for monitoring the distribution of the antibody within the body.

As used herein, “physiologically compatible carrier” refers to a physiologically acceptable diluent such as water, phosphate buffered saline, or saline. Pharmaceutically acceptable camera are well known in the art (see for example Remington's Pharmaceutical Sciences, 16th Edition, 1980, Mac Publishing Company). Such pharmaceutically acceptable carriers may include other medicinal agents, carriers, genetic carriers, adjuvants, excipients, etc., such as human serum albumin or plasma preparations. Adjuvants such as incomplete Freund's adjuvant, aluminum phosphate, aluminum hydroxide, or alum are materials well known in the art.

The invention also provides for pharmaceutical compositions. In addition to the active ingredients, these pharmaceutical compositions may contain suitable pharmaceutically acceptable carrier preparations which can be used pharmaceutically.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the subject.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, and related acids. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a Ph range of 4.5 to 5.5 that is combined with buffer prior to use.

After pharmaceutical compositions comprising a compound of the invention formulated in a acceptable carrier have been prepared, they can be placed in an appropriate container and labeled for treatment of an indicated condition with information including amount, frequency and method of administration.

### Dosage and Mode of Administration

The pharmaceutical compositions are preferably in the form of a unit dose and will usually be administered one or more times a day.

If formulated as a fixed dose, such combination products employ the compounds of this invention within an appropriate dosage range as determined by the skilled practitioner, and the other pharmaceutically active agent(s) within its approved dosage range. Compounds of the instant invention may alternatively be used sequentially with known pharmaceutically acceptable agent(s) when a combination formulation is inappropriate.

The pharmaceutical compositions of this invention may also be administered using microspheres, liposomes, other microparticulate delivery systems or sustained release formulations placed in, near, or otherwise in communication with affected tissues or the bloodstream. Suitable examples of sustained release carriers include semipermeable polymer matrices in the form of shaped articles such as suppositories or microcapsules. Implantable or microcapsular sustained release matrices include polylactides (U.S. Pat. No. 3,773,319; EP 58,481), copolymers of L-glutamic acid and gamma ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

Liposomes containing beta1-integrin or FAK inhibitors can be prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of beta1-integrin or FAK inhibitor release.

The beta 1-integrin or FAK inhibitors of this invention may also be attached to liposomes, which may optionally contain other agents to aid in targeting or administration of the compositions to the desired treatment site. Attachment of beta1-integrin or FAK inhibitors to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

The beta1-integrin or FAK inhibitors of this invention may be formulated into pharmaceutical compositions and administered in vivo at an effective dose to treat the particular clinical condition addressed. Administration of one or more of the pharmaceutical compositions according to this invention will be useful for regulating and for promoting neural growth or regeneration in the nervous system, for treating injuries or damage to nervous tissue or neurons, and for treating neural degeneration associated with traumas to the nervous system, disorders or diseases. Such traumas, diseases or disorders include, but are not limited to: aneurysms, strokes, Alzheimer's disease, Parkinson's disease, Creutzfeldt-Jacob disease, kuru, Huntington's disease, multiple system atrophy, amyotropic lateral sclerosis (Lou Gehrig's disease), and progressive supranuclear palsy.

Determination of a preferred pharmaceutical formulation and a therapeutically efficient dose regiment for a given application is within the skill of the art taking into consideration, for example, the condition and weight of the patient, the extent of desired treatment and the tolerance of the patient for the treatment.

Administration of the beta1-integrin or FAK inhibitors of this invention, including isolated and purified forms, their salts or pharmaceutically acceptable derivatives thereof, may be accomplished using any of the conventionally accepted modes of administration of agents which are used to treat neuronal injuries or disorders.

Soluble altered and mutated forms of beta 1-integrin or FAK such as those described herein are prepared from the culture media of transfected cells. The soluble molecules, such as beta1-integrin-Fc or FAK-Fc, are secreted by these cells. It is anticipated that, as has been carried out for hybridoma cells that secrete antibodies (Schnell, L. and Schwab, M. E., Nature, 343, pp. 269-72 (1990); Schnell et al., Nature, 367, pp. 170-73 (1993), COS cells or other transfectants secreting the soluble beta1-integrin-Fc or FAK-Fc chimera may be implanted into damaged spinal cord. The cells will secrete beta1-integrin- or FAK-inhibiting forms of altered or mutated beta1-integrin or FAK-Fc, which perturbs activity of the endogenous beta1-integrin or FAK. Transfected cells, secreting other “reversing” mutated forms of beta 1-integrin or FAK or beta1-integrin or FAK “blocking” peptides can be administered to the site of neuronal injury or degeneration in a similar manner.

Likewise, other beta1-integrin or FAK inhibitors and regulators of this invention can also be delivered by spinal implantation (e.g., into the cerebrospinal fluid) of cells engineered to secrete the beta 1-integrin or FAK regulating agents according to this invention. Cell secretion rates of the agent are measured in cell culture and then extrapolated.

Optionally, transfected cells that secrete beta1-integrin or FAK regulating agents may be encapsulated into immunoisolatory capsules or chambers and implanted into the brain or spinal cord region using available methods that are known to those of skill in the art. See, e.g., WO 89/04655; WO 92/19195; WO 93/00127; EP 127, 989; U.S. Pat. No. 4,298,002; U.S. Pat. No. 4,670,014; U.S. Pat. No. 5,487,739 and references cited therein, all of which are incorporated herein by reference.

For beta1-integrin or FAK regulating agents that can not be secreted by transfected cells, a pump and catheter-like device may be implanted at the site of injury to administer the agent on a timely basis and at the desired concentration, which can be selected and empirically modified by one of skill in the art. Such pharmaceutical delivery systems are known to those of skill in the art. See, e.g., U.S. Pat. No. 4,578,057 and references cited therein, which are incorporated herein by reference.

The pharmaceutical compositions of this invention may be in a variety of forms, which may be selected according to the preferred modes of administration. These include, for example, solid, semi-solid and liquid dosage forms such as tablets, pills, powders, liquid solutions or suspensions, suppositories, and injectable and infusible solutions. The preferred form depends on the intended mode of administration and therapeutic application. Modes of administration may include oral, parenteral, subcutaneous, intravenous, intralesional or topical administration.

The beta1-integrin or FAK inhibitors of this invention may, for example, be placed into sterile, isotonic formulations with or without cofactors which stimulate uptake or stability. The formulation is preferably liquid, or may be lyophilized powder. For example, the beta1-integrin or FAK inhibitors may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

In some cases the beta1-integrin or FAK inhibitor may be a RNA or DNA molecule. RNA or siRNA containing medicaments may contain substances which stabilize double-stranded RNA molecule and/or which enable the double-stranded RNA molecule or DNA expression vector to be transfected or to be injected into the human or animal cell.

Administration can be carried out by known methods, wherein a nucleic acid is introduced into a desired cell in vitro or in vivo. For therapeutic applications, the medicament may be in form of a solution, in particular an injectable solution, a cream, ointment, tablet, suspension, granulate or the like. The medicament may be administered in any suitable way, in particular by injection, or by oral, nasal or rectal application. The medicament may particularly be administered parenterally, that means without entering the digestion apparatus, for example by subcutaneous injection. The medicament may also be injected intravenously in the form of solutions for infusions or injections. Other suitable administration forms may be direct administrations on the skin in the form of creams, ointments, sprays and other transdermal therapeutic substances or in the form of inhalative substances, such as nose sprays, aerosols or in the form of microcapsules or implantates.

The optimal administration form and/or administration dose for a medicament either comprising double-stranded RNA molecules with the above sequences or comprising nucleic acid vectors capable to express such double-stranded RNA molecules depend on the type and the progression of the disease to be treated, and can be determined by the skilled practitioner.

### Combination Therapies

The beta1-integrin inhibitor or the FAK inhibitor can be administered in combination with, or prior or subsequent to, other treatment regimes such as the use of anti-inflammatory agents. For example, a neural lesion may result from acute spinal cord injury. The combination treatment method may additionally comprise contacting the neuron with an anti-inflammatory agent. Exemplary anti-inflammatory agents include Nonsteroidal anti-inflammatory drugs (also called NSAIDs), for example COX-2 inhibitors. Compounds which are described as specific inhibitors of COX-2 and are therefore useful in the present invention, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999. Other exemplary anti inflammatories that can be administered with the compositions of the instant invention include corticosteroids. For example, methylprednisolone is a corticosteroid that can be administered for reduction of CNS inflammation. In another embodiment, the condition results from neurodegeneration which, for example, can be caused by neurotoxicity or a neurological disease or disorder such as Huntington's disease, Parkinson's disease, Alzheimer's disease, multiple system atrophy (MSA). Current treatment regimes used for the above-mentioned diseases may be used in combination with the compositions of the invention.

Further, known inhibitors of FAK and/or beta1-integrin may be used with the compositions of the invention. For instance, integrin blockers may be used to selectively antagonize, inhibit or counter-act binding of a physiological ligand to the integrin receptor complex. FAK blockers may be used to selectively antagonize, inhibit or counter-act activation of FAK. Further, blocking antibodies, siRNA/RNAi, shRNA, microRNA, antisense oligonucleotides, ribozymes, aptamers, chemical agents, phosphorylation inhibitors, or small molecules may be administered as a second agent with the compositions of the invention.

### Kits

The present compositions may be assembled into kits or pharmaceutical systems for use in treatment of a disease or disorder of the CNS or PNS. Kits or pharmaceutical systems according to this aspect of the invention comprise a carrier means, such as a box, carton, tube or the like, having in close confinement therein one or more container means, such as vials, tubes, ampules, bottles and the like. The kits or pharmaceutical systems of the invention may also comprise associated instructions for using the compounds of the invention for treating or preventing MAG-induced growth inhibition of neural cells and/or promoting the growth of neural cells, or a MAG-induced disease or disorder of the CNS or PNS. The kits or pharmaceutical systems of the invention may also comprise any combination of agents for treating or preventing MAG-induced growth inhibition of neural cells and/or promoting the growth of neural cells, or a MAG-induced disease or disorder of the CNS or PNS, and a second therapeutic agent, and instructions for use.

### Examples

It should be appreciated that the invention should not be construed to be limited to the examples that are now described; rather, the invention should be construed to include any and all applications provided herein and all equivalent variations within the skill of the ordinary artisan.

### Example I

### Interaction Between Myelin Associated Glycoprotein (MAG) and β1-Integrin

To determine whether β1-integrin interacts with myelin associated glycoprotein (MAG), cultured primary hippocampal neurons were treated with recombinant MAG consisting of the MAG extracellular domain fused to human Fc (MAG-Fc; see Experimental Procedures), a fusion protein previously shown to potently regulate neurite outgrowth and induce growth cone turning responses (Domeniconi et al., 2002; Henley et al., 2004; Liu et al., 2002; McKerracher et al., 1994; Shim et al., 2005; Song et al., 1998). MAG and β1-integrin were co-immunoprecipitated with either antibodies directed against β-integrin or human Fc, as shown in FIGS. 1B and 1C, suggesting that they interact with one another. This association between MAG and β1-integrin was attenuated by the disintegrin echistatin, a viper-venom-derived RGD peptide that specifically inhibits β1 and β3 containing integrins (Gan et al., 1988), and by the specific β1-integrin function-blocking antibody Ha2/5 (Mendrick and Kelly, 1993) (FIG. 1D).

To directly examine the requirement of the ROD motif for MAG association with β1-integrin, a mutant form of MAG that is not recognized by integrins (Yip et al., 1998), in which the RGD motif was mutated to KGE (MAG-KGE; FIG. 1A) was used. This mutant form is termed MAG-KGE. Purified MAG-KGE was unable to interact with β1-integrin, whereas purified MAG-RGD (the wild-type) was able to interact under these same conditions, as shown in FIG. 1C. These results suggest that the association between MAG and β1-integrin is direct, and occurs via a classical mode of integrin-ligand interaction (Ruoslahti, 1996; Ruoslahti and Pierschbacher, 1987).

### Example 2

### Requirement of β1-Integrin Function in MAG-Induced Acute Growth Cone Turning

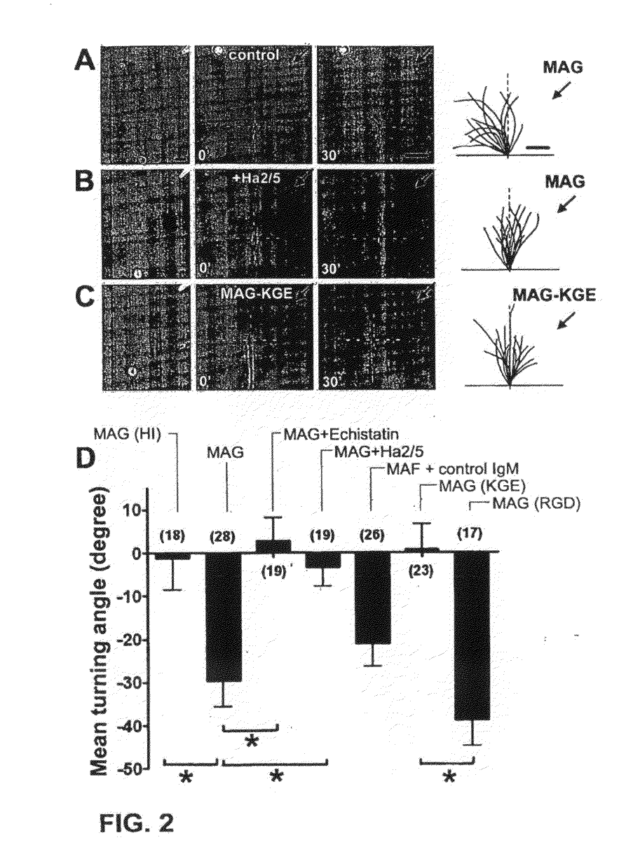

To examine the functional role of β1-integrin in transducing MAG signaling in neuronal growth cones, growth cone turning assays were performed using rat hippocampal neurons. Growth cone turning assays are well-known and described in the art, for example by Song et al., 1998; Xiang et al., 2002; see also Experimental Procedures. Axonal growth cones of postnatal day 5 (P5) hippocampal neurons exhibited acute repulsive turning responses in a microscopic gradient of recombinant MAO (150 μg/ml in the pipette), as shown in FIG. 2A, while the hippocampal neurons showed no bias in the direction of axonal extension with heat-inactivated MAG (MAG-HI), as shown in FIG. 2D. MAG-induced growth cone repulsion was completely abolished in the presence of 100 nM echistatin or 1.0 μml Ha2/5 (FIGS. 2B and 2D), but not in the presence of a control IgM (FIG. 2D). Furthermore, a gradient of mutant MAG lacking an intact RGD motif (MAG-KGE) failed to induce significant repulsion of these hippocampal neurons (FIG. 2D). Thus, this data indicates that β1-integrin function is required for MAG-induced axonal growth cone repulsion.

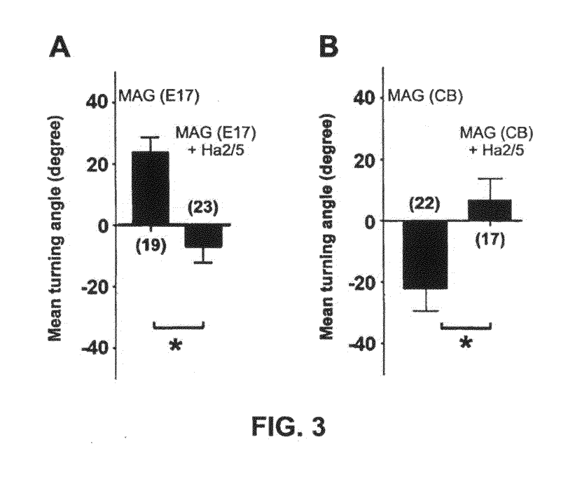

It is known that MAG promotes neurite outgrowth during embryonic development (Cai et al., 2001; Johnson et al., 1989; Mukhopadhyay et al., 1994; Turnley and Bartlett, 1998). Thus, MAG-induced turning responses of embryonic hippocampal neurons was next examined. MAG-induced significant attractive turning responses of axonal growth cones in embryonic day 17 (E17) rat hippocampal neurons, as shown in FIG. 3A. Interestingly, this MAG-induced attraction was also abolished in the presence of Ha2/5 (FIG. 3A), indicating that β1-integrin function in both MAG-induced attraction and repulsion of hippocampal neurons of different developmental stages.

To determine whether MAG-integrin interactions are limited to hippocampal neurons, growth cone turning responses of rat cerebellar neurons was examined. A MAG gradient (150 μg/ml in the pipette) induced significant growth cone repulsion of these neurons, as shown in FIG. 3B. Importantly, MAG-induced repulsion of cerebellar neurons was also abolished in the presence of Ha2/5 (FIG. 3B). Taken together, these results show that β1-integrin function is required for MAG-induced acute growth cone steering in different types of mammalian neurons.

### Example 3

### β1-Intergin Function is not Required for OMgp-Induced Growth Cone Turning

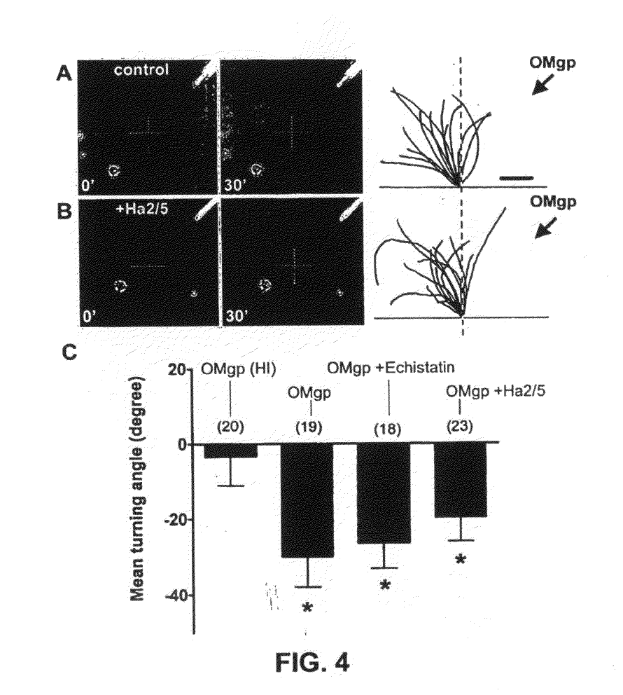

Three major myelin-associated inhibitory factors, Nogo-66, MAG and OMgp, are known to bind the common NgR protein, and may utilize the same signal transduction pathway to inhibit axonal extension (Domeniconi et al., 2002; Fournier et al., 2001; Liu et al., 2002; Wang et al., 2002b). Therefore, whether β1-integrin also mediates growth cone responses to other myelin-associated inhibitors was next considered. Axonal growth cones of rat P5 hippocampal neurons exhibited significant repulsive turning responses to a gradient of recombinant OMgp (5 μg/ml in the pipette), but not to heat-inactivated Omgp, as shown in FIGS. 4A and 4B. In contrast to observations for MAG, axonal growth cones still exhibited significant repulsion to OMgp in the presence of echistatin or Ha2/5, as shown in FIG. 4C. Thus, β1-integrin appears to specifically mediate acute axonal growth cone turning induced by MAG, but not by OMgp.

### Example 4

### β1-Integrin Mediates MAG-Induced Growth Cone Turning Independent of NgR

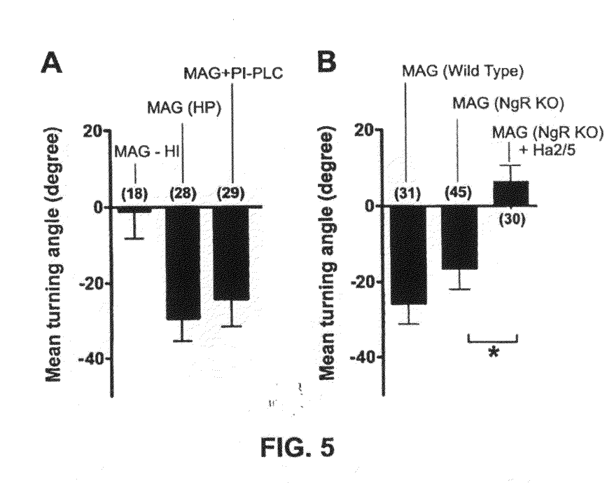

To address whether β1-integrin provides an independent pathway for mediating MAG signaling or acts as a co-receptor along with NgR/p75/TROY/Lingo-1, turning responses following removal of GPI-linked proteins, including NgRs, from the neuronal cell surface were next examined. Primary hippocampal neurons were pre-treated with phosphatidylinositol-phopholipase C(PI-PLC) (1 unit/ml) for 30 min at 37 C (Liu et al., 2002; Wang et al., 2002b), and growth cones were examined in a gradient of MAG (150 μg/ml in the pipette) with the continuous presence of PI-PLC (See Experimental Procedures). Under these conditions, axonal growth cones of P5 rat hippocampal neurons still exhibited significant repulsive responses to MAG, as shown in FIG. 5A. These results are consistent with previous findings that MAG retains its ability to induce RhoA activation in P7 cerebellar granule cells following the PI-PLC treatment (Niederost et al., 2002), and that mutant forms of MAG-Fc lacking an intact RGD domain (AGD or DGD) lose their inhibitory activities on axonal extension of cultured cerebellar neurons (Tang et al., 1997a).

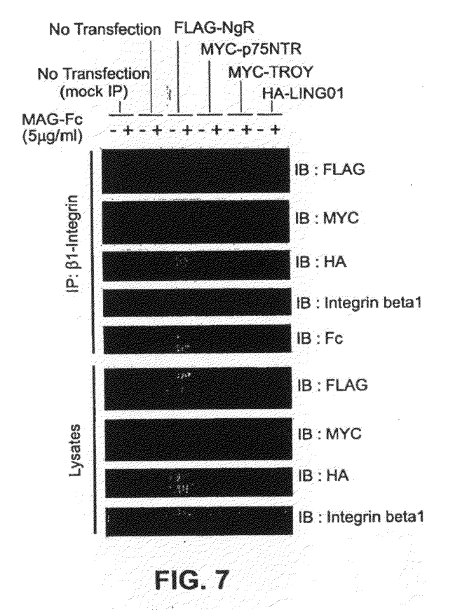

To directly assess the role of NgR in MAG-induced acute growth cone repulsion, neurons from NgR −/− mice (Zheng et al., 2005) were examined. Hippocampal neurons lacking NgR still exhibited significant repulsive turning responses to the MAG gradient (150 μg/ml in the pipette; FIG. 5B), suggesting that NgR is dispensible for MAG-induced growth cone repulsion. More importantly, MAG-induced repulsion of neurons lacking NgR was also abolished by 1.0 μg/ml Ha2/5 (FIG. 5B). In addition, there were no detectable interactions between 131-integrin and any member of the known NgR signaling complex, including NgR, p75, TROY and Lingo-1, either in the presence or absence of MAG, as shown in FIG. 7. Taken together, these findings are consistent with the notion that β1-integrin mediates MAG-induced acute growth cone turning responses independent of the known NgR receptor complex.

### Example 5

### FAK Mediates MAG-Induced Growth Cone Turning Downstream of β1-Integrin

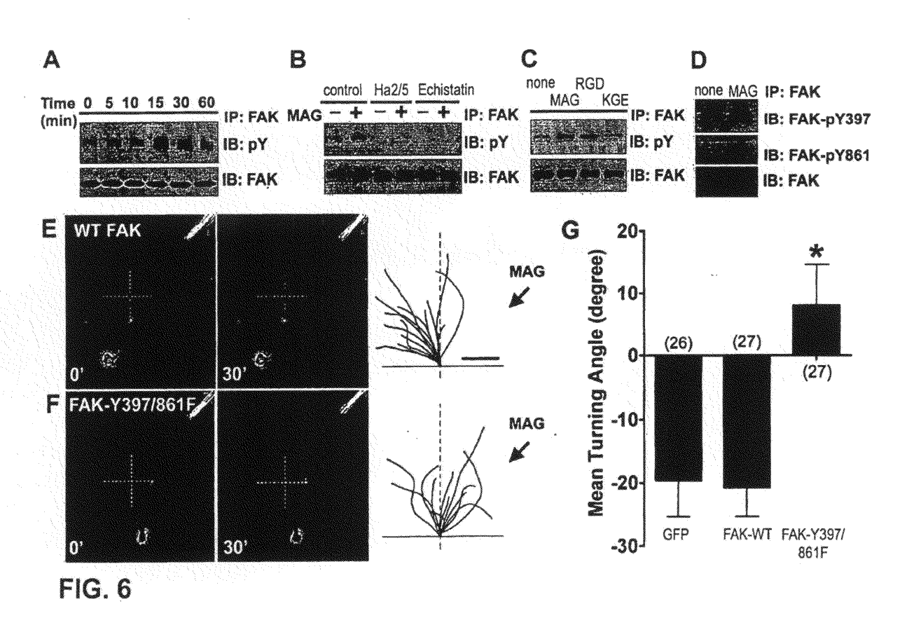

The question was next asked how β1-integrin signaling transduces MAG-induced neuronal responses. Focal adhesion kinase (FAK) is a major mediator of integrin-dependent signaling in many contexts, including cell migration and axon guidance (Nakamoto et al., 2004; Nikolopoulos and Gianotti, 2005; Xie and Tsai, 2004). Treatment of hippocampal neurons with MAG (2 μg/ml) was found to induce FAK tyrosine phosphorylation in a time-dependent manner, as shown in FIG. 6A. MAG-induced FAK phosphorylation was abolished in the presence of echistatin (100 nM) or Ha2/5 (2.0 μg/ml) (FIG. 6B). In addition, mutant MAG-KGE failed to trigger tyrosine phosphorylation of FAK (FIG. 6C). However, PI-PLC treatment did not affect MAG-induced FAK phosphorylation in these neurons (FIG. 6). Thus, the results indicate that MAG induces FAK phosphorylation in an integrin-dependent manner. The specific FAK tyrosine residues that become phosphorylated upon MAG stimulation in hippocampal neurons were next examined. Using site-specific phospho-tyrosine FAK antibodies, we found that MAG induced a significant increase in the phosphorylation of FAK tyrosine residues 397 and 861, as shown in FIG. 6D. Similar β1-integrin-dependent FAK phosphorylation of these tyrosine residues was also found in embryonic cortical neurons treated with MAG (data not shown).

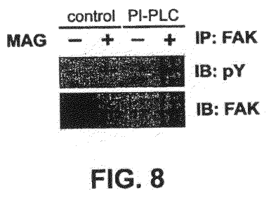

To determine the functional role of FAK tyrosine phosphorylation in MAG-induced growth cone turning, P5 rat hippocampal neurons were transfected with either wild-type (WT) FAK or a mutant FAK (FAK-Y397/861F) that cannot be phosphorylated on tyrosine residues 397 and 861 (See Experimental Procedures). Expression of mutant FAK-Y397/861F, but not WT-FAK, abolished MAG-induced repulsion, as shown in FIGS. 8E to 8G. Together, these findings demonstrate that MAG-induced FAK phosphorylation functions in growth cone turning responses to MAG and indicate that FAK is a downstream component that mediated integrin signaling triggered by MAG.

Taken together, the results presented herein indicated that β1-integrin acts as a functional receptor to mediate MAG-induced acute turning responses at axonal growth cones and promotion of long-term axonal stability at axonal shafts of several neuronal types. Previous studies led to the finding that Nogo66, OMgp and MAG, three major inhibitors associated with myelin, all bind to NgR and appear to signal at axonal growth cones through a common receptor complex containing NgR, p75/TROY and Lingo-1 (Brittis and Flanagan, 2001; Filbin, 2003; Mandemakers and Barres, 2005; Yiu and He, 2006). Two additional human homologs for NgR (NgR2 and NgR3) are found to be expressed in CNS neurons (Lauren et al., 2003; Pignot et al., 2003). While neither binds to Nogo66 (Barton et al., 2003), NgR2 appears to bind to MAG (Venkatesh et al., 2005). Accumulating evidence suggests that inhibitors associated with myelin may signal independently of NgRs (Niederost et al., 2002; Zheng et al., 2005). MAG has also been reported to inhibit neurite outgrowth through a sialoglycoprotein (DeBellard et al., 1996; Tang et al., 1997a) and gangliosides (Vyas et al., 2002) in postnatal DRG neurons and cerebellar granule neurons. MAG signaling at the axon shaft was previously unknown. In vivo findings indicate that NgR does not mediate MAO-dependent protection against progressive spontaneous axonal degeneration with age in both PNS and CNS. Instead, our studies show that integrin signaling through FAK serves as a common mediator for MAG-induced effects at both axonal growth cones and shafts. It is likely that additional downstream signaling components determine the differential effects induced by MAG at neuronal growth cones of distinct developmental stages and at axonal shafts. For example, we have shown that growth cone turning responses to netrin-1 or Sema3A lead to either attraction or repulsion utilizing the same guidance receptor mechanisms, but depending on cytoplasmic levels of cyclic nucleotides (Ming et al., 1997; Song et al., 1998).

The results show that 31-integrin is a mediator for MAG, but not for OMgp. Consistent with a selective involvement of β1-integrin in mediating MAG effects, human and rodent MAG contain a RGD-tri-peptide motif characteristic of integrin binding proteins (Ruoslahti, 1996; Ruoslahti and Pierschbacher, 1987), whereas OMgp and Nogo do not. Interestingly, MAG homologs in fugu and Zebrafish, species with the capacity for axonal regeneration, do not contain an intact RGD motif (FIG. 1A). The extent to which different receptors mediate distinct effects of MAG in various species remains to be determined. Our results demonstrate that integrin/FAK signaling provides an alternative pathway for mediating MAG effects independent of the NgR receptor complex.

### MATERIALS AND METHODS OF THE INVENTION

The results reported herein were obtained using the following Materials and Methods:

### Primary Neuronal Cultures and Neuronal Transfection

Hippocampal neurons were isolated from embryonic and postnatal rats, or wild-type and NgR knockout mice (Zheng et al., 2005) as previously described (Song et al., 2002a; Song et al., 2002b). Similarly, cerebellar neurons were isolated from P5 rat cerebellum (Xiang et al., 2002). They were cultured on poly-L-lysine coated plates or coverslips as previously described (Song et al., 2002a; Song et al., 2002b). For biochemical analysis, embryonic day 18 (E18) neurons were treated with the agent AraC to eliminate dividing astrocytes and used at 5 days after plating as previously described (Song et al., 2002a; Song et al., 2002b). For growth cone turning assay, neurons were used between 2-3 days after plating. PI-PLC (1 or 2 units/ml) (Wang et al., 2002b), Ha2/5 (1 μg/ml) or echistatin (100 nM) (Gan et al., 1988) were added for 30 mins prior to and were present during the biochemical analysis or the growth cone turning assay.

Rat primary hippocampal neurons were transfected using reagent supplied in the Amaxa transfection system following protocols from the manufacturer. Briefly, hippocampal neurons were isolated and 100 μl of nucleofector solution was added to resuspend the cell pellet. Different expression constructs (1-5 μg), GFP, WT-FAK-GFP or FAK-Y397/861F-GFP (Ren et al., 2004), were added to the cell suspension and the cell-DNA mix was then transferred to cuvettes for electroporation. The cells were cultured in DMEM with 10% fetal bovine serum for 24 hrs before changing to serum-free neurobasal medium (Song et al., 2002a; Song et al., 2002b).

### Constructs and Biochemistry

Mutation of MAG-Fc was generated by site directed mutagenesis. Expression plasmids of wild-type MAG (ROD) or mutant MAG (KGE) were transfected into 293 Ebna cells and proteins were collected from the media and affinity purified using protein A sepharose. Neurons at 5 days after plating were treated with 2 units/ml PI-PLC, 100 nM Echistatin or 0.5-2.0 μg/ml Ha2/5 and stimulated with 2.0 μg/ml MAG or 0.5 μg/ml OMgp for the indicated time periods. Cells were then lysed in immunoprecipitation buffer (1% Triton X-100; 150 mM NaCl; 10 mM Tris, pH 7.4; 1 mM EDTA; 1 mM EGTA; 1% Nonidet P-40; 0.2 mM Na₃VO4; 1 μg/ml protease inhibitor cocktail; and 0.1 mM PMSF). Samples were immunoprecipitated with polyclonal antibody against FAK (Santa Cruz Biotechnology, Inc.), human Fc (Sigma) or β1-integrin (Chemicon.) and then subjected to Western Blot analysis. The following antibodies were used: monoclonal antibody against tyrosine phosphorylated proteins (pY20, Transduction Laboratories; 1:1000), and rabbit polyclonal antibodies against β1-integrin (1:1000), FAK (1:1000), FAK-pY397 (Biosource; 1:1000), FAK-pY861 (Biosource; 1:1000), or human Fc (1:1000). Blots were stripped and reblotted with the same antibodies used for their immunoprecipitation to ensure equal loading of the immunoprecipitated proteins. For experiments testing potential interactions between β1-integrin and the NgR receptor complex, HEK293 cells were transfected with cDNA for NgR, p75, TROY, or Lingo-1, respectively, as previously described (Park et al., 2005). Transfected cells were simulated with MAG or medium and then were immunoprecipitated with anti-β1-integrin antibodies and immunoblotted for respective components of the NgR receptor complex. Total cell lysates were also examined to show the expression of endogenous β1-integrin and proteins from transfection.

### Growth Cone Turning Assay

Microscopic gradients of recombinant MAG (150 μg/ml in the pipette) and OMgp (5 μg/ml in the pipette) were produced as previously described (Lohof et al., 1992; Song et al., 1998; Xiang et al., 2002). Axons were identified as the longest neurite in these cultures at the stage 2-3 of hippocampal neurons as previously described (Dotti et al., 1988). Growth cone assays were carried out at room temperature for 30 min. The turning angle was defined by the angle between the original direction of neurite extension and a straight line connecting the position of the center of the growth cone at the onset and the end of the 30 min period. Only those with axonal extension >5 μm over the 30 minute period were included for analysis. Statistical significance was assessed using ANOVA and accepted if p<0.01.

### FAK Dominant Negative

FAK dominant negative mutant was generated using the Quick change kit (stratagene) by changing the tyrosine residue at position 397 and 861 to phenylanaline. A GFP tag was fused at the end as an indicator. FAK-Y397/861 F-GFP was then electroporated into the cells and the expression of this mutant was identified by GFP signal.

### INCORPORATION BY REFERENCE

The contents of all references, patents, pending patent applications and published patents, cited throughout this application are hereby expressly incorporated by reference.

### LITERATURE CITED

Barton, W. A., Liu, B. P., Tzvetkova, D., Jeffrey, P. D., Fournier, A. E., Sah, D., Cate, R., Strittmatter, S. M., and Nikolov, D. B. (2003). Structure and axon outgrowth inhibitor binding of the Nogo-66 receptor and related proteins. Embo J 22, 3291-3302.
Brittis, P. A., and Flanagan, J. G. (2001). Nogo domains and a Nogo receptor: implications for axon regeneration. Neuron 30, 11-14.
Cai, D., Qiu, J., Cao, Z., McAtee, M., Bregman, B. S., and Filbin, M. T. (2001). Neuronal cyclic AMP controls the developmental loss in ability of axons to regenerate. J Neurosci 21, 4731-4739.
Dashiell, S. M., Tanner, S. L., Pant, H. C., and Quarles, R. H. (2002). Myelin-associated glycoprotein modulates expression and phosphorylation of neuronal cytoskeletal elements and their associated kinases. J Neurochem 81, 1263-1272.
Davy, A., and Robbins, S. M. (2000). Ephrin-A5 modulates cell adhesion and morphology in an integrin-dependent manner. Embo J 19, 5396-5405.
de Waegh, S. M., Lee, V. M., and Brady, S. T. (1992). Local modulation of neurofilament phosphorylation, axonal caliber, and slow axonal transport by myelinating Schwann cells. Cell 68, 451-463.
DeBellard, M. E., Tang, S., Mukhopadhyay, G., Shen, Y. J., and Filbin, M. T. (1996). Myelin-associated glycoprotein inhibits axonal regeneration from a variety of neurons via interaction with a sialoglycoprotein. Mol Cell Neurosci 7, 89-101.
Domeniconi, M., Cao, Z., Spencer, T., Sivasankaran, R., Wang, K., Nikulina, E., Kimura, N., Cai, H., Deng, K., Gao, Y., et al. (2002). Myelin-associated glycoprotein interacts with the Nogo66 receptor to inhibit neurite outgrowth. Neuron 35, 283-290.
Dotti, C. G., Sullivan, C. A., and Banker, G. A. (1988). The establishment of polarity by hippocampal neurons in culture. J Neurosci 8, 1454-1468.
Filbin, M. T. (2003). Myelin-associated inhibitors of axonal regeneration in the adult mammalian CNS. Nat Rev Neurosci 4, 703-713.
Fournier, A. E., GrandPre, T., and Strittmatter, S. M. (2001). Identification of a receptor mediating Nogo-66 inhibition of axonal regeneration. Nature 409, 341-346.
Gan, Z. R., Gould, R. J., Jacobs, J. W., Friedman, P. A., and Polokoff, M. A. (1988). Echistatin. A potent platelet aggregation inhibitor from the venom of the viper, Echis carinatus. J Biol Chem 263, 19827-19832.
Gomez, T. M., Robles, E., Poo, M., and Spitzer, N. C. (2001). Filopodial calcium transients promote substrate-dependent growth cone turning. Science 291, 1983-1987.
He, Z., and Koprivica, V. (2004). The Nogo signaling pathway for regeneration block. Annu Rev Neurosci 27, 341-368.
Henley, J. R., Huang, K. H., Wang, D., and Poo, M. M. (2004). Calcium mediates bidirectional growth cone turning induced by myelin-associated glycoprotein. Neuron 44, 909-916.
Hynes, R. O. (2002). Integrins: bidirectional, allosteric signaling machines. Cell 110, 673-687.
Johnson, P. W., Abramow-Newerly, W., Seilheimer, B., Sadoul, R., Tropak, M. B., Arquint, M., Dunn, R. J., Schachner, M., and Roder, J. C. (1989). Recombinant myelin-associated glycoprotein confers neural adhesion and neurite outgrowth function. Neuron 3, 377-385.
Kim, J. E., Liu, B. P., Park, J. H., and Strittmatter, S. M. (2004). Nogo-66 receptor prevents raphespinal and rubrospinal axon regeneration and limits functional recovery from spinal cord injury. Neuron 44, 439-451.
Lauren, J., Airaksinen, M. S., Saarma, M., and Timmusk, T. (2003). Two novel mammalian Nogo receptor homologs differentially expressed in the central and peripheral nervous systems. Mol Cell Neurosci 24, 581-594.
Lee, D. H., Strittmatter, S. M., and Sah, D. W. (2003). Targeting the Nogo receptor to treat central nervous system injuries. Nat Rev Drug Discov 2, 872-878.
Liu, H. P., Fournier, A., GrandPre, T., and Strittmatter, S. M. (2002). Myelin-associated glycoprotein as a functional ligand for the Nogo-66 receptor. Science 297, 1190-1193.
Lohof, A. M., Quillan, M., Dan, Y., and Poo, M. M. (1992). Asymmetric modulation of cytosolic cAMP activity induces growth cone turning. J Neurosci 12, 1253-1261.
Mandemakers, W. J., and Barres, B. A. (2005). Axon regeneration: it's getting crowded at the gates of TROY. Curr Biol 15, R302-305.
May, A. P., Robinson, R. C., Vinson, M., Crocker, P. R., and Jones, E. Y. (1998). Crystal structure of the N-terminal domain of sialoadhesin in complex with 3′ sialyllactose at 1.85 A resolution. Mol Cell 1, 719-728.
McKerracher, L., David, S., Jackson, D. L., Kottis, V., Dunn, R. J., and Braun, P. E. (1994). Identification of myelin-associated glycoprotein as a major myelin-derived inhibitor of neurite growth. Neuron 13, 805-811.
Mendrick, D. L., and Kelly, D. M. (1993). Temporal expression of VLA-2 and modulation of its ligand specificity by rat glomerular epithelial cells in vitro. Lab Invest 69, 690-702.
Mi, S., Lee, X., Shao, Z., Thill, G., Ji, B., Relton, J., Levesque, M., Allaire, N., Perrin, S., Sands, B., et al. (2004). LINGO-1 is a component of the Nogo-66 receptor/p75 signaling complex. Nat Neurosci 7, 221-228.
Ming, G. L., Song, H. J., Berninger, B., Holt, C. E., Tessier-Lavigne, M., and Poo, M. M. (1997). cAMP-dependent growth cone guidance by netrin-1. Neuron 19, 1225-1235.
Mukhopadhyay, G., Doherty, P., Walsh, F. S., Crocker, P. R., and Filbin, M. T. (1994). A novel role for myelin-associated glycoprotein as an inhibitor of axonal regeneration. Neuron 13, 757-767.
Nakamoto, T., Kain, K. H., and Ginsberg, M. H. (2004). Neurobiology: New connections between integrins and axon guidance. Curr Biol 14, R121-123.
Niederost, B., Oertle, T., Fritsche, J., McKinney, R. A., and Bandtlow, C. E. (2002). Nogo-A and myelin-associated glycoprotein mediate neurite growth inhibition by antagonistic regulation of RhoA and Rac1. J Neurosci 22, 10368-10376.
Nikolopoulos, S, N., and Giancotti, F. G. (2005). Netrin-integrin signaling in epithelial morphogenesis, axon guidance and vascular patterning. Cell Cycle 4, e131-135.
Pan, B., Fromholt, S. E., Hess, E. J., Crawford, T. O., Griffin, J. W., Sheikh, K. A., and Schnaar, R. L. (2005). Myelin-associated glycoprotein and complementary axonal ligands, gangliosides, mediate axon stability in the CNS and PNS: neuropathology and behavioral deficits in single- and double-null mice. Exp Neurol 195, 208-217.
Park, J. B., Yiu, G., Kaneko, S., Wang, J., Chang, J., He, X. L., Garcia, K. C., and He, Z. (2005). A TNF receptor family member, TROY, is a coreceptor with Nogo receptor in mediating the inhibitory activity of myelin inhibitors. Neuron 45, 345-351.
Pasterkamp, R. J., Peschon, J. J., Spriggs, M. K., and Kolodkin, A. L. (2003). Semaphorin 7A promotes axon outgrowth through integrins and MAPKs. Nature 424, 398-405.
Pedraza, L., Owens, G. C., Green, L. A., and Selzer, J. L. (1990). The myelin-associated glycoproteins: membrane disposition, evidence of a novel disulfide linkage between immunoglobulin-like domains, and posttranslational palmitylation. J Cell Biol 111, 2651-2661.
Pignot, V., Hein, A. E., Barske, C., Wiessner, C., Walmsley, A. R., Kaupmann, K., Mayeur, H., Sommer, B., Mir, A. K., and Frentzel, S. (2003). Characterization of two novel proteins, NgRH1 and NgRH2, structurally and biochemically homologous to the Nogo-66 receptor. J Neurochem 85, 717-728.
Ren, X. R., Ming, G. L., Xie, Y., Hong, Y., Sun, D. M., Zhao, Z. Q., Feng, Z., Wang, Q., Shim, S., Chen, Z. F., et al. (2004). Focal adhesion kinase in netrin-1 signaling. Nat Neurosci 7, 1204-1212.
Ridley, A. J., Schwartz, M. A., Burridge, K., Firtel, R. A., Ginsberg, M. H., Borisy, G., Parsons, J. T., and Horwitz, A. R. (2003). Cell migration: integrating signals from front to back. Science 302, 1704-1709.
Ruoslahti, E. (1996). RGD and other recognition sequences for integrins. Annu Rev Cell Dev Biol 12, 697-715.
Ruoslahti, E., and Pierschbacher, M. D. (1987). New perspectives in cell adhesion: RGD and integrins. Science 238, 491-497.
Sadoul, R., Fahrig, T., Bartsch, U., and Schachner, M. (1990). Binding properties of liposomes containing the myelin-associated glycoprotein MAG to neural cell cultures. J Neurosci Res 25, 1-13.
Schachner, M., and Bartsch, U. (2000). Multiple functions of the myelin-associated glycoprotein MAG (siglec-4a) in formation and maintenance of myelin. Glia 29, 154-165.
Schafer, M., Fruttiger, M., Montag, D., Schachner, M., and Martini, R. (1996). Disruption of the gene for the myelin-associated glycoprotein improves axonal regrowth along myelin in C57BL/W1ds mice. Neuron 16, 1107-1113.
Serini, G., Valdembri, D., Zanivan, S., Morterra, G., Burkhardt, C., Caccavari, F., Zammataro, L., Primo, L., Tamagnone, L., Logan, M., et al. (2003). Class 3 semaphorins control vascular morphogenesis by inhibiting integrin function. Nature 424, 391-397.
Shao, Z., Browning, J. L., Lee, X., Scott, M. L., Shulga-Morskaya, S., Allaire, N., Thill, G., Levesque, M., Sah, D., McCoy, J. M., et al. (2005). TAJ/TROY, an orphan TNF receptor family member, binds Nogo-66 receptor 1 and regulates axonal regeneration. Neuron 45, 353-359.
Shim, S., Goh, E. L., Ge, S., Sailor, K., Yuan, J. P., Roderick, H. L., Bootman, M. D., Worley, P. F., Song, H., and Ming, G. L. (2005). XTRPC1-dependent chemotropic guidance of neuronal growth cones. Nat Neurosci 8, 730-735.
Song, H., Ming, G., He, Z., Lehmann, M., McKerracher, L., Tessier-Lavigne, M., and Poo, M. (1998). Conversion of neuronal growth cone responses from repulsion to attraction by cyclic nucleotides. Science 281, 1515-1518.
Song, H., Stevens, C. F., and Gage, F. H. (2002a). Astroglia induce neurogenesis from adult neural stem cells. Nature 417, 39-44.
Song, H. J., Stevens, C. F., and Gage, F. H. (2002b). Neural stem cells from adult hippocampus develop essential properties of functional CNS neurons. Nat Neurosci 5, 438-445.
Stevens, A., and Jacobs, J. R. (2002). Integrins regulate responsiveness to slit repellent signals. J Neurosci 22, 4448-4455.
Tang, S., Shen, Y. J., DeBellard, M. E., Mukhopadhyay, G., Salzer, J. L., Crocker, P. R., and Filbin, M. T. (1997a). Myelin-associated glycoprotein interacts with neurons via a sialic acid binding site at ARG118 and a distinct neurite inhibition site. J Cell Biol 138, 1355-1366.
Tang, S., Woodhall, R. W., Shen, Y. J., deBellard, M. E., Saffell, J. L., Doherty, P., Walsh, F. S., and Filbin, M. T. (1997b). Soluble myelin-associated glycoprotein (MAG) found in vivo inhibits axonal regeneration. Mol Cell Neurosci 9, 333-346.
Turnley, A. M., and Bartlett, P. F. (1998). MAG and MOG enhance neurite outgrowth of embryonic mouse spinal cord neurons. Neuroreport 9, 1987-1990.
Venkatesh, K., Chivatakarn, O., Lee, H., Joshi, P. S., Kantor, D. B., Newman, B. A., Mage, R., Rader, C., and Giger, R. J. (2005). The Nogo-66 receptor homolog NgR2 is a sialic acid-dependent receptor selective for myelin-associated glycoprotein. J Neurosci 25, 808-822.
Vyas, A. A., Patel, H. V., Fromholt, S. E., Heffer-Lauc, M., Vyas, K. A., Dang, J., Schachner, M., and Schnaar, R. L. (2002). Gangliosides are functional nerve cell ligands for myelin-associated glycoprotein (MAG), an inhibitor of nerve regeneration. Proc Natl Acad Sci USA 99, 8412-8417.
Wang, K. C., Kim, J. A., Sivasankaran, R., Segal, R., and He, Z. (2002a). P75 interacts with the Nogo receptor as a co-receptor for Nogo, MAG and OMgp. Nature 420, 74-78.
Wang, K. C., Koprivica, V., Kim, J. A., Sivasankaran, R., Guo, Y., Neve, R. L., and He, Z. (2002b). Oligodendrocyte-myelin glycoprotein is a Nogo receptor ligand that inhibits neurite outgrowth. Nature 417, 941-944.
Wong, S. T., Henley, J. R., Kanning, K. C., Huang, K. H., Bothwell, M., and Poo, M. M. (2002). A p75(NTR) and Nogo receptor complex mediates repulsive signaling by myelin-associated glycoprotein. Nat Neurosci 5, 1302-1308.
Xiang, Y., Li, Y., Zhang, Z., Cui, K., Wang, S., Yuan, X. B., Wu, C. P., Poo, M. M., and Duan, S. (2002). Nerve growth cone guidance mediated by G protein-coupled receptors. Nat Neurosci 5, 843-848.
Xie, Z., and Tsai, L. H. (2004). Cdk5 phosphorylation of FAK regulates centrosome-associated miocrotubules and neuronal migration. Cell Cycle 3, 108-110.
Yebra, M., Montgomery, A. M., Diaferia, G. R., Kaido, T., Silletti, S., Perez, B., Just, M. L., Hildbrand, S., Hurford, R., Florkiewicz, E., et al. (2003). Recognition of the neural chemoattractant Netrin-1 by integrins alpha6beta4 and alpha3beta1 regulates epithelial cell adhesion and migration. Dev Cell 5, 695-707.
Yin, X., Crawford, T. O., Griffin, J. W., Tu, P., Lee, V. M., Li, C., Roder, J., and Trapp, B. D. (1998). Myelin-associated glycoprotein is a myelin signal that modulates the caliber of myelinated axons. J Neurosci 18, 1953-1962.
Yip, P. M., Zhao, X., Montgomery, A. M., and Siu, C. H. (1998). The Arg-Gly-Asp motif in the cell adhesion molecule L1 promotes neurite outgrowth via interaction with the alphavbeta3 integrin. Mol Biol Cell 9, 277-290.
Yiu, G., and He, Z. (2006). Glial inhibition of CNS axon regeneration. Nat Rev Neurosci 7, 617-627.
Zaccai, N. R., Maenaka, K., Maenaka, T., Crocker, P. R., Brossmer, R., Kelm, S., and Jones, E. Y. (2003). Structure-guided design of sialic acid-based Siglec inhibitors and crystallographic analysis in complex with sialoadhesin. Structure (Camb) 11, 557-567.
Zheng, B., Atwal, J., Ho, C., Case, L., He, X. L., Garcia, K. C., Steward, O., and Tessier-Lavigne, M. (2005). Genetic deletion of the Nogo receptor does not reduce neurite inhibition in vitro or promote corticospinal tract regeneration in vivo. Proc Natl Acad Sci USA 102, 1205-1210.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for inhibiting myelin associated glycoprotein (MAG)-induced growth inhibition in a neural cell, the method comprising contacting a neural cell with an effective amount of a beta1-integrin inhibitor or focal adhesion kinase (FAK) inhibitor, thereby inhibiting myelin associated glycoprotein (MAG)-induced growth inhibition in neural cells.

2. The method of claim 1, wherein MAG-induced growth inhibition in the neural cell comprises inhibition of axonal regeneration.

3. The method of claim 1, wherein the method increases neural growth, axonal outgrowth, or axonal regeneration.

4-5. (canceled)

6. The method of claim 5, wherein the trauma or disease is selected from the group consisting of aneurysm, stroke, spinal cord or brain injury, Alzheimer's disease, Parkinson's disease, Creutzfeldt-Jacob disease, kuru, Huntington's disease, multiple system atrophy, amyotropic lateral sclerosis, spinal muscular atrophy, and progressive supranuclear palsy.

7. The method of claim 1, wherein the method comprises administering a beta1-integrin inhibitor and a focal adhesion kinase (FAK) inhibitor.

8. The method of claim 1, wherein the beta1-integrin inhibitor or focal adhesion kinase (FAK) inhibitor is a small compound, blocking antibody, polypeptide, polynucleotide, dominant negative polypeptide, or fragment thereof.

9. A method for promoting neural cell growth, the method comprising contacting a neural cell with an effective amount of a beta1-integrin inhibitor or focal adhesion kinase (FAK) inhibitor, thereby promoting neural cell growth.

10. The method of claim 9, wherein the neural cell is in contact with MAG.

11. A method for promoting neural cell growth in the presence of MAG, contacting the neural cell with a therapeutically effective amount of a beta1-integrin inhibitor or focal adhesion kinase (FAK) inhibitor, thereby promoting the growth of the neural cell in the presence of MAG.

12. (canceled)

13. A method for inhibiting myelin associated glycoprotein (MAG) induced growth inhibition, the method comprising:
identifying a patient suffering from or susceptible to MAG-induced growth inhibition; and
administering a beta1-integrin inhibitor or FAK inhibitor compound to the identified patient, thereby inhibiting MAG-induced growth inhibition.

14. (canceled)

15. A method for promoting the growth of a neural cell, the method comprising:
identifying a patient suffering from or susceptible to a MAG-induced inhibition of axonal regeneration; and
administering a beta1-integrin inhibitor or FAK inhibitor compound to the identified patient, thereby promoting the growth of neural cells.

16. A method of treating or preventing a MAG-induced disease or disorder of the nervous system, the method comprising:
identifying a patient suffering from or susceptible to a MAG-induced disease or disorder of the nervous system; and
administering to the identified patient a beta1-integrin inhibitor or FAK inhibitor compound, thereby treating the disease or disorder.

17-33. (canceled)

34. A method for identifying an agent that inhibits MAG-induced growth inhibition in neural cells, the method comprising:
contacting a neural cell with a candidate agent;
detecting altered neural cell growth; and
selecting the agent as a candidate if neural cell growth is promoted; thereby identifying an agent that inhibits myelin associated glycoprotein (MAG)-induced growth inhibition.

35-45. (canceled)

46. A pharmaceutically acceptable composition comprising an agent that inhibits MAG-induced growth inhibition in neural cells, and a pharmaceutically acceptable carrier or (ii) a FAK inhibitor and/or a beta1 integrin inhibitor.

47-49. (canceled)

50. A kit comprising one or more agents that inhibits MAG-induced growth inhibition of neural cells and promotes the growth of neural cells, and instructions for use.

51-52. (canceled)
